(19) United States
(12) Patent Application Publication
(10) Pub. No.: US 2009/0099484 A1
(43) Pub. Date: Apr. 16, 2009

(54) COATING OF POLYURETHANE MEMBRANE

(76) Inventors: Yehuda Zadok, Holon (IL); Avner Yanai, Ganei Tikva (IL)

Correspondence Address: EMPK & Shiloh, LLP;c/o Landon IP, Inc., 1700 Diagonal Road, Suite 450, Alexandria, VA, 22314, US

(21) Appl. No.: 11/870,521

(22) Filed: Oct. 11, 2007

(51) Int. Cl. A61N 7/00; B05D 3/00

(52) U.S. Cl. 601/2; 427/2.1

(57) ABSTRACT

There is provided a vibration delivery system that includes one or more vibrating elements, a vibration focusing element and a protective coating on said focusing element.

### BACKGROUND

The field of aesthetic medicine is a fast growing area in which medical procedures as well as medical devices are used to promote aesthetic traits. One of the most popular areas in the aesthetic medical field is the removal and/or reduction of the number of subcutaneous fat cells and volume of adipose tissue. Removal and/or reduction of the number of subcutaneous fat cells and the volume of adipose tissue may result in the reshaping of body parts, frequently referred to as “body contouring”.

To date, various techniques have been proposed to aid in the task of lowering the number and/or volume of fat cells and adipose tissue. One of the most widely used such technique is liposuction. Liposuction is a medical procedure that involves surgical removal of all or part of the subcutaneous fat cell layer in target areas of the body. This procedure is invasive and involves local or general anesthesia. The procedure involves the insertion of, for example, a cannule through a small skin incision into the adipose tissue whereby the fat is then suctioned out. The cannule may be moved back and forth in different tissue levels covering the volume to be suctioned. The fat is torn and evacuated at the same time. This procedure may require several incisions to be made to the skin and is non selective, as along with fat tissues, other surrounding tissues, such as blood vessels, nerves and connective tissues may tear. Side effects of this procedure are hematomas, hypo-sensation and pain and recovery time may be prolonged.

A related procedure to liposuction is Ultrasound Assisted Lipoplasty (UAL). UAL uses a cannule that has an ultrasound probe at its tip when energy is applied; the tissue next to the tip is destroyed by effect known as “cavitational effect”. The fat that is destroyed by the procedure may be evacuated by the same, or another cannule. Common side effects of this method are skin, muscle or bone tissue damage, skin burns and the like.

An additional technique, known as External Ultrasound Assisted Liposuction (EUAL) employs the usual liposuction technique but adds a treatment with a therapeutic ultrasonic transducer. The ultrasound treatment is applied after a tumescent solution is injected into the subject. The energy which is applied by this method may involve heating of the skin and underlying tissue, which may result in damage to tissue.

In addition to the methods mentioned aboveherein, various other fat removal techniques and procedure have been described, such as, for example, use of medications, ointments, laser based procedures, radio frequency (RF) based procedures, ultrasound based procedures and the like.

Among the ultrasound-based procedures for fat and adipose tissue removal, an additional body contouring solution involves a non-invasive treatment. The non-invasive treatment is based on the application of focused therapeutic ultrasound that selectively targets and disrupts fat cells without damaging neighboring structures. This may be achieved by, for example, a device, such as a transducer, that delivers focused ultrasound energy to the subcutaneous fat layer. Specific, pre-set ultrasound parameters ensure that only the fat cells within the treatment area are targeted and that neighboring structures such as blood vessels, nerves and connective tissue remain intact. Since ultrasonic energy may considerably be attenuated in air, in order to efficiently transmit ultrasonic energy to a subcutaneous fat layer, an interposer substance having appropriate acoustic impedance may be placed between the subject body and the device that delivers the focused ultrasound energy.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to some embodiments, there is provided a vibration delivery system that includes one or more vibrating elements, a vibration focusing element and a protective coating on the focusing element.

According to some embodiments, the protective coating is a chemically protective coating that may be composed of PVC coating or of polyolefin film. The coating may be applied by adhering to the focusing element or by mechanical application. According to further embodiments, the coating may be acoustically matched to the focusing element.

According to some embodiments, the focusing element may include a polyurethane membrane.

According to some embodiments, there is provided a coating for a vibration focusing element that includes a protective material adapted to inhibit a reaction between the focusing element and an interposer.

According to some embodiments, the protective material is a chemically protective material and the reaction is a chemical reaction.

According to some embodiments, the interposer may be a lubricant and may include castor oil.

According to further embodiments, the protective material may include PVC coating or a polyolefin film. The protective material may be applied by adhering to the focusing element or by mechanical application.

According to some embodiments, there is provided a vibration delivery system that includes one or more vibrating elements, an acoustic coupling interface and a protective coating on the acoustic coupling interface.

According to some embodiments, acoustic coupling interface may include polyurethane membrane.

According to some embodiments, the protective coating is a chemically protective coating, that may be composed of PVC coating or of polyolefin film. The coating may be applied by adhering to the acoustic coupling interface or by mechanical application. According to further embodiments, the coating may be acoustically matched to the acoustic coupling interface.

According to some embodiments, there is provided a coating for an acoustic coupling interface that includes a protective material adapted to inhibit a reaction between the acoustic coupling interface and an external surface.

According to some embodiments, the external surface may include a substrate, a tissue, a substance, an interposer, or any combination thereof.

According to some embodiments, the protective material is a chemically protective material, and the reaction is a chemical reaction.

According to some embodiments, the tissue may include a skin tissue, an adipose tissue, or any combination thereof.

According to some embodiments, the interposer may be a lubricant and may include castor oil.

According to further embodiments, the protective material may include PVC coating or a polyolefin film. The protective material may be applied by adhering to the focusing element or by mechanical application.

According to further embodiments there is provided a system for selectively damaging fat cells, that includes at least one vibrating energy transducer, an acoustic coupling interface and a protective coating on said acoustic coupling interface. The protective coating may be a chemically protective coating.

According to some embodiments, there is provided a method for ameliorating chemical interaction between an acoustic coupling interface and an interposer, the method comprising applying a chemically protective coating on said acoustic coupling interface. The interposer may include a lubricant, such as castor oil.

In addition to the exemplary aspects and embodiments described above, further aspects and embodiments will become apparent by reference to the figures and by study of the following detailed descriptions.

### BRIEF DESCRIPTION OF THE FIGURES

FIG. 1—a schematic block diagram of a transducing system according to some embodiments;

FIG. 2—an illustration of a perspective side view of a transducer according to some embodiments;

FIG. 3—a close up perspective side of a generator end of a connecting component of a transducer, according to some embodiments;

FIG. 4—an illustration of internal close-up side view of a connection component of a transducer, according to some embodiments;

FIG. 5—an illustration of an internal perspective view of a transducing part, according to some embodiments;

FIG. 6—an illustration of a perspective view of the internal parts of transducing box, according to some embodiments;

FIG. 7—an illustration of a perspective view of a cross section of a transducing part, according to some embodiments;

FIG. 8—an illustration of a perspective cross-section view of an acoustic coupling interface, according to some embodiments;

FIG. 9—an illustration of a simplified schematic drawing of an experimental setup to test transducer related parameters, according to some embodiments.

### DETAILED DESCRIPTION

In the following description, various aspects of the invention will be described. For the purpose of explanation, specific configurations and details are set forth in order to provide a thorough understanding of the invention. However, it will also be apparent to one skilled in the art that the invention may be practiced without specific details being presented herein. Furthermore, well-known features may be omitted or simplified in order not to obscure the invention.

According to some embodiments, there is provided a vibration delivery system that includes one or more vibrating elements, a vibration focusing element and a protective coating on the focusing element. The vibration delivery system may include a transducer that may transduce vibration energy such as ultrasound energy. The ultrasound energy may be focused and may be used, for example, to selectively target and disrupt fat cells without damaging neighboring tissues in a subject body.

As referred to herein, the term a “subject body” may include all or any part of a subject body, both internally and/or externally. For example, a “subject body” may include, an entire body; body part, such as a limb; an organ, such as for example, a liver; a tissue, such as for example, skin tissue, subcutaneous adipose tissue, blood vessel, nerve tissue and the like; cells, such as, for example, fat cells, blood cells, and the like. The term working surface and user body may interchangeably be used.

As referred to herein, the terms transducer, transducer unit, transducing unit, therapeutic transducer, vibration delivery system may interchangeably be used.

As referred to herein, the terms acoustic energy, acoustic waves, ultrasonic energy, ultrasonic waves, vibration energy, vibration waves, may interchangeably be used.

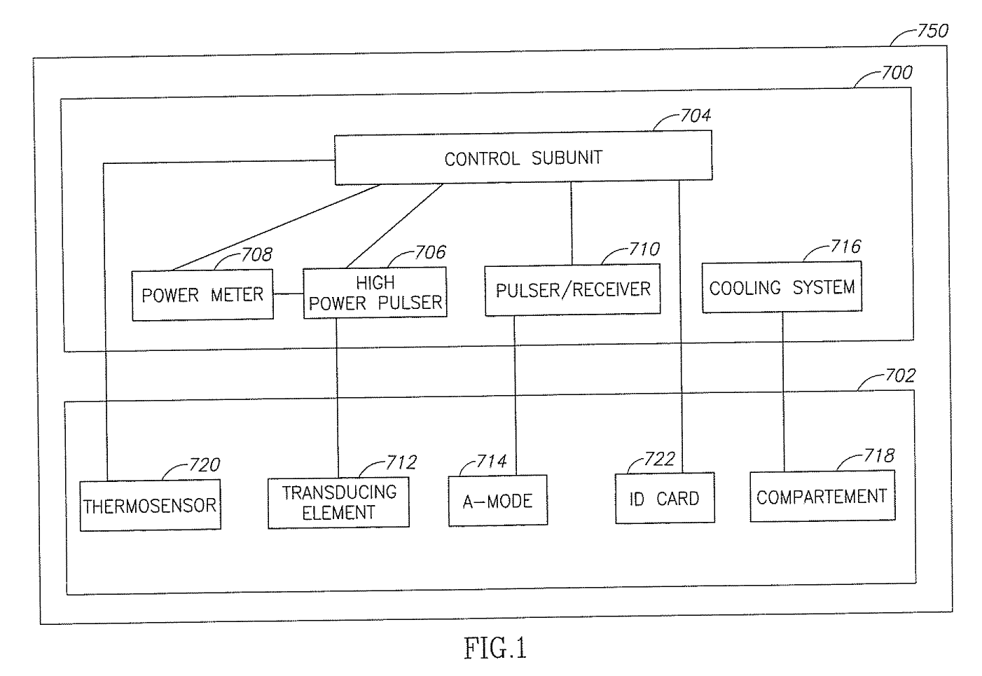

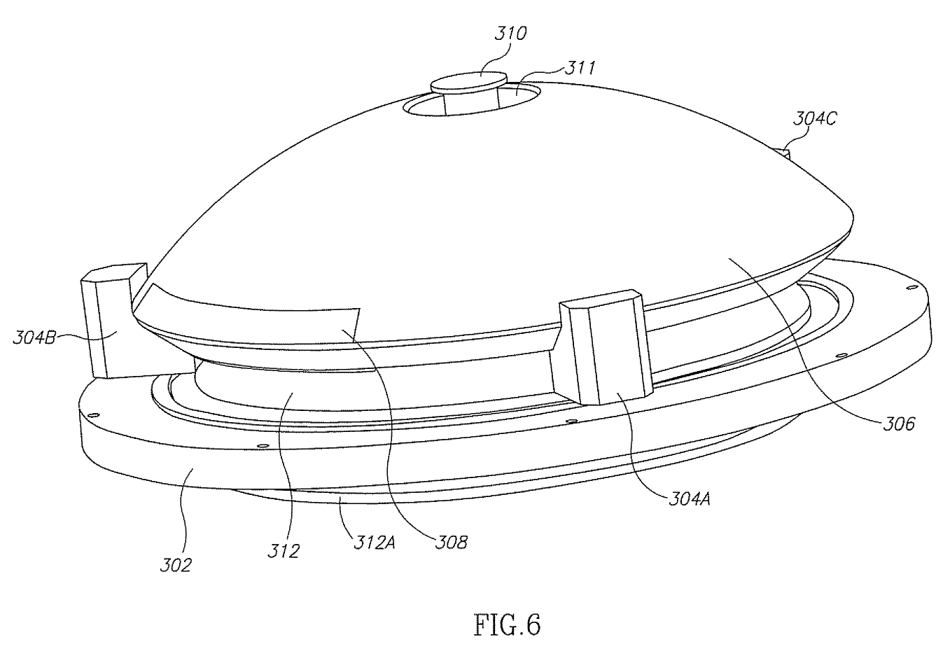

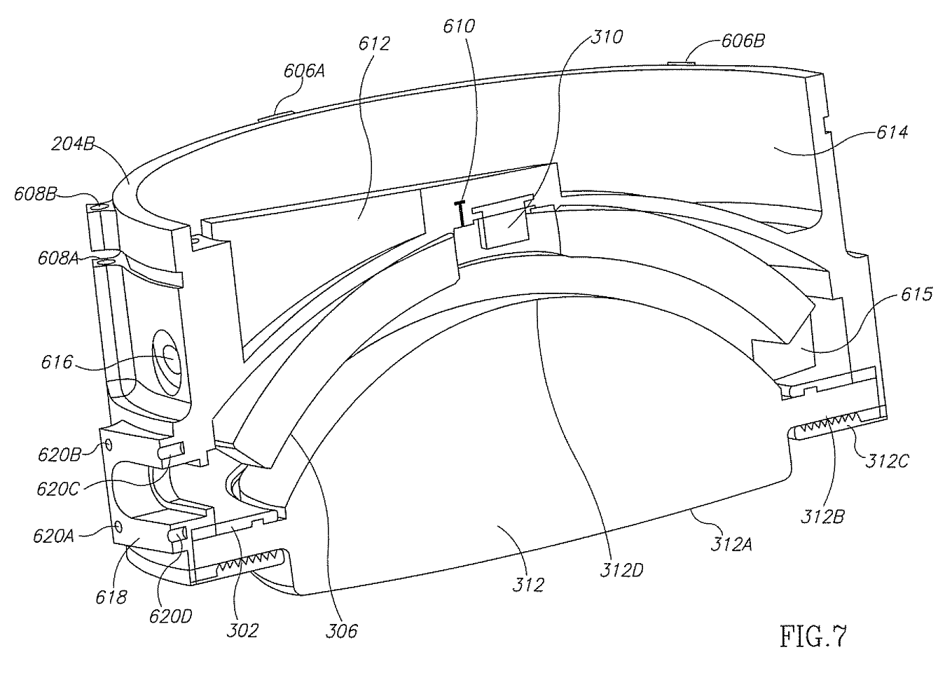

Reference is now made to FIG. 1, which illustrates a schematic block diagram of a transducing system according to some embodiments. The transducing system (750) may include a generator unit, 700 and a transducer unit, 702. Generator unit 700 may provide the transducer unit with, for example power, energy, fluids, software instruction, control and feedback information and the like. Generator unit 700 may include several subunits that may be independent and/or interconnected and may be individually or commonly controlled. Generator unit 700 may include a control subunit 704. Control subunit 704 may include any appropriate hardware and software that may be used to control and coordinate operation of various components of the transducing system. For example, control subunit 704 may include electronic circuits, processors, ROM and RAM memories and the like. Control subunit 704 may receive and send information from the various components of the transducing system. Generator unit 700 may include several power sources. For example, high power pulser, 706, may provide power to the transducer unit. In particular, high power pulser may provide power to the transducing element ((712), further detailed below), located within the transducer unit. Power pulser 706 may provide pulses of power to the transducer unit. The pulses may be in the duration of, for example about 0.1 to 10 seconds. The pulses may be in the duration of, for example about 0.1 to 8 seconds. The pulses may be in the duration of, for example 0.1 to 6 seconds. The pulses may be in the duration of, for example 0.1 to 4 seconds. The pulses may be in the duration of, for example 0.1 to 3 seconds. The pulses may be in the duration of, for example 0.1 to 2 seconds. The pulses may be in the duration of, for example 0.1 to 1 seconds. The pulses may be in the duration of for example 0.1 to 0.5 seconds. Power meter 708 may be used for power measurement and control of high power pulser 706. Pulser/receiver 710 may be used to provide power to the transducer unit. In particular, pulser/receiver 710 may be used to provide power to the A-mode transducer ((714), further detailed herein below), located within the transducer unit. In addition, a cooling system 716 may be included and/or attached to the generator unit. Cooling system may be attached to the transducer unit and aid in cooling environment in the transducer unit. Cooling system 716 may include, for example, a circulating cooling system, that may provide inflow and outflow of cooling fluids, such as water, between the cooling system and a compartment 718 (detailed herein below) in the transducer unit (702). The transducer unit (702) may further include thermosensor(s) (720) that may include any kind of one or more thermosensors that may be used to measure temperature at various regions of the transducer. The thermosensors (720) may further be connected to the control unit (704) to transfer and receive information. Transducing element 710 may be used to produce vibration energy, such as in the form of acoustic energy. Furthermore, the acoustic energy produced by the transducing element may be focused. (A detailed description of transducing element is followed herein below, with respect to FIGS. 6-7) A-mode transducer 714 may be used as an ultrasonic probe and may be used to provide assessment of acoustic contact between the transducer unit and a subject to which the acoustic energy is transducer (A detailed description of A-mode transducer is followed herein below, with respect to FIGS. 6-7). In addition, the transducer unit may include an ID card (Chip) (722). ID Card 722 may include any kind of relevant information regarding the transducer unit, such as, for example, serial number, specifications, mode of action, length of operation and the like. ID card 722 may further send and receive information to control subunit (704). The use of ID card 722 may be used to monitor use of the transducer unit and coordinate operation of the transducer unit with the generator unit. For example, after a preset number of pulses have been delivered by the transducer unit (such as in the range of 12000-50000), the control subunit (704) may prevent further use of the transducer. As further detailed herein below, connection between the various subunits and elements of the generator unit and the transducer unit may be performed by use of various connectors, cables, wires, pipes, and the like and may also be performed by wireless means.

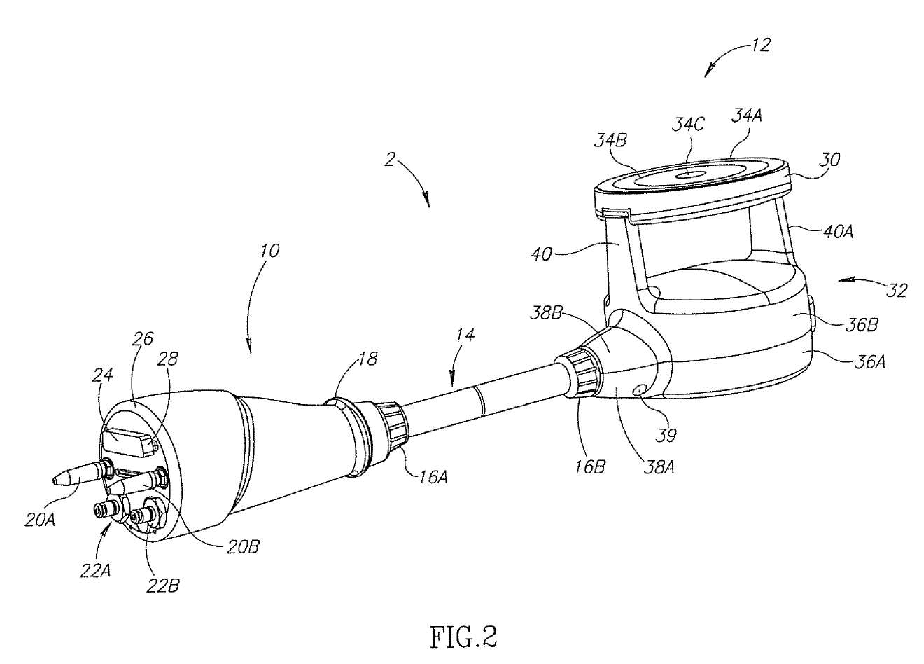

Reference is now made to FIG. 2, which illustrates a perspective side view of a transducer according to some embodiments. As shown in FIG. 2, the transducer, 2, may include two components that are located at two ends of the transducer: the “connection component” (10) and the “vibration component” (12). The two components may be connected by a bridging element (14). The connection component, 10, may include elements that may be used to connect the transducer to additional devices such as a Generator unit 700 (FIG. 1) that may provide the transducer with power, energy, fluids, software instruction, control and feedback, and the like. The vibration component, 12 may be used to produce, concentrate and output vibration energy and may come in close proximity and/or contact with a subject body.

The bridging element, (such as bridging element 14 in FIG. 2), may include any bridging element that may be used to physically and/or functionally connect and/or assist in connecting and/or bridging the components (connection component 10 and vibration component, 12) of transducer 2. The bridging element may include, for example, a hollow bridging element, a flat bridging element, a virtual bridging element or any combination thereof. The bridging element may be flexible, elastic, semi-flexible, rigid, stretchable, or any combination thereof and may be comprised of one continuous element, or non-continuous elements that may be connected to each other. The length of the bridging element may be fixed or changeable, such as for example by stretching a stretchable bridging element. The bridging element may further include, at its ends, fitting elements that may be used to physically connect and/or secure the bridging element to the components of the transducer, as detailed herein below. The bridging element may be constructed of various materials that may include, for example plastic, rubber, metal and the like, and may further be coated with such materials. The bridging element may further be adapted to carry and protect various additional constituents that may interact with the both ends of the transducer, as detailed below herein. According to some embodiments, the bridging element may include a continuous elastic, flexible plastic tube coated with a rubber coating so as to externally protect the tube. The bridging tube may be at least partially hollow so as to allow a protected transfer/transmission of various other constituents, such as cables, wires (for example, electrical cables and wires, hardware cables and wires, and the like), pipes (such as air pipes, fluid pipes and the like), tubes (such as air tubes, fluid tubes and the like) that may run through the bridging element, between the components of the transducer. In such a manner, the various other constituents that may be connected between the components of the transducer are protected within the bridging element. The bridging element may be secured to the components of the transducer by various ways, such as for example, by the use of screws, bolts, nuts, hinges, pressure, and the like. As shown by way of example in FIG. 2, the bridging element, 14, may be connected to the two components of the transducer by the use of screwable nuts, such as nuts 16A and 16B. The nuts may be constructed of metal, plastic or any combination thereof and may be screwed on to a perforated helix that may be an integral part of the bridging element (such as bridging element 14), the vibration component (12), the connection component (10), the various additional constituents that may interact with the both ends of the transducer (not shown), or any combination thereof.

According to further embodiments, the bridging element may be a virtual element. A virtual bridging element may include such elements that do not necessarily have a tangible presence, but may be used to functionally connect between the components (such as the vibration component (12) and the connection component (10)) of the transducer. Functionally connecting between the transducer components may be performed, for example, by the transfer of information and/or energy between the transducer components. For example, the virtual bridging element may include a wireless communication route between the components of the transducer.

Reference is now made to connection component (10) illustrated in FIG. 2. The connection component, 10, may include two opposing ends. One end, the “generator end” is the end that may connect to the generator unit. The second, opposing end, referred to herein as the “bridging end” is the end that may connect to the bridging element. The connection component, 10, may have a non-symmetrical spherical tube-like shape, having a broader diameter at the generator end, and whereby at about a third of the way towards the bridging end the diameter is becoming continuously narrower. At about an eight way of the bridging end, the connection component may include a ring like shape, such as ring 18, that may be an integral part of the connection component. The connection component may include at least a partially hollow interior, contained within the tube-like shape that may be comprised of one integral part or of at least two parts that are interconnected. At the generator end of the connection component, a cover plate, such as cover plate 26, may be attached to the connection component body, 10. Cover plate 26, may be secured to the connection component body, 10, by the use of screws, such as screw 28 in FIG. 2. The cover plate may be secured to the connection component body, so as to seal the generator end of the connection component. On the face of the cover plate, several connectors of various kinds may be located. The connectors may protrude out of the cover plate, towards the generator end of the connection component. The connectors may be used to physically and functionally connect the connecting component to a generator unit. According to some embodiments, and as shown in FIG. 2 by way of example, the connectors may include such connectors as connectors 20A and 20B, that may be, for example, structural connectors (pins), earth connectors and the like; connectors 22A and 22B that may be, for example, fluid connectors (such as air, water, oil and the like) and connector 24, that may include a D-type connector, power connectors, I/O connectors and the like.

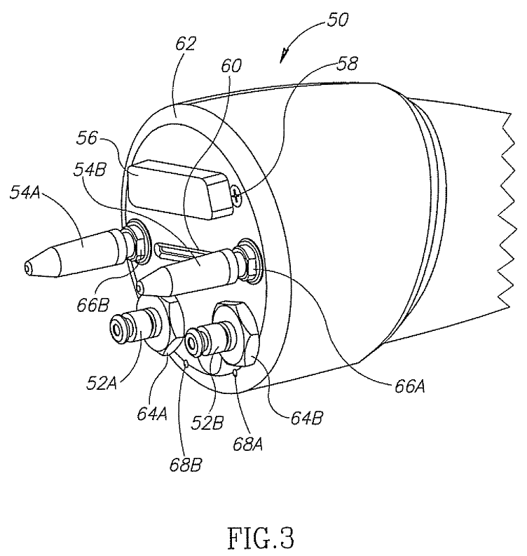

Reference is now made to FIG. 3, which illustrates a close up perspective side view of the generator end of a connecting component of a transducer, according to some embodiments. The connecting component, 50, may include a cover plate, such as cover plate 62 that may have an external diameter that is similar to the diameter of the broad part of the connection component, so that the cover plate may seal the generator end of the connection component. The cover plate, 62, may be secured to the generator end of the connection component by various ways, such as screws, nuts, pressure, and the like. For example, the cover plate may be secured to the connection component 50, by the use of screws, such as screw 58 in FIG. 3. Further shown in FIG. 3 are various connectors that may be located on cover plate 62. The connectors may be permanently mounted onto the cover plate, or may be reversibly connected to the plate by ways such as pressure, screws, nuts and the like. The connectors may be user accessible and replaceable. For example, connectors 52A and 52B may be identified at the bottom part of cover plate 62. Connectors, such as connectors 52A and 52B may be used to connect transiently or permanently to pipes, tubes and the like and may be used for the transmittal of fluids such as fluids at their gaseous state or at their liquid state of aggregation, as, for example water. Connectors 52A and 52B may be identical or different and may be connected to identical or different pipes, tubes and the like. Connectors, such as connectors 52A and 52B, may be transiently connected to the outer side (generator side) of the cover plate by the use of nuts, such as nuts 64A and 64B, respectively. Connectors, such as connectors 54A and 54B, may be identified at about the center region of the cover plate. Connectors, 54A and 54B may be fixed to the cover plate, for example by nuts 66A and 66B, respectively. Connectors 54A and 54B may be used for example as structural connectors, pins, alignment connectors, earth (grounding) connectors and the like. Connectors 54A and 54B may further be used for the transmittal of energy, such as for example electrical energy. At the top region of cover plate, connector, such as connector 56 may be identified. Connector 56 may be mounted on the cover plate and protrude out towards the generator end. Connector, such as connector 56 may include such connectors as D-type connector, power connectors, I/O connectors and the like that may be used for the transmittal of digital and/or analog information. Holes, such as pinholes 68A and 68B may be identified at the bottom region of the cover plate. Holes 68A and 68B may be used for ventilation purposes and/or for the emergency dismantling of the cover plate from the connection component. In addition, slot, such as slot 60 in FIG. 3 may also be found on the cover plate. Slots or perforations, such as slot 60, may be to ensure proper alignment between the covet plate and the connection component, 50.

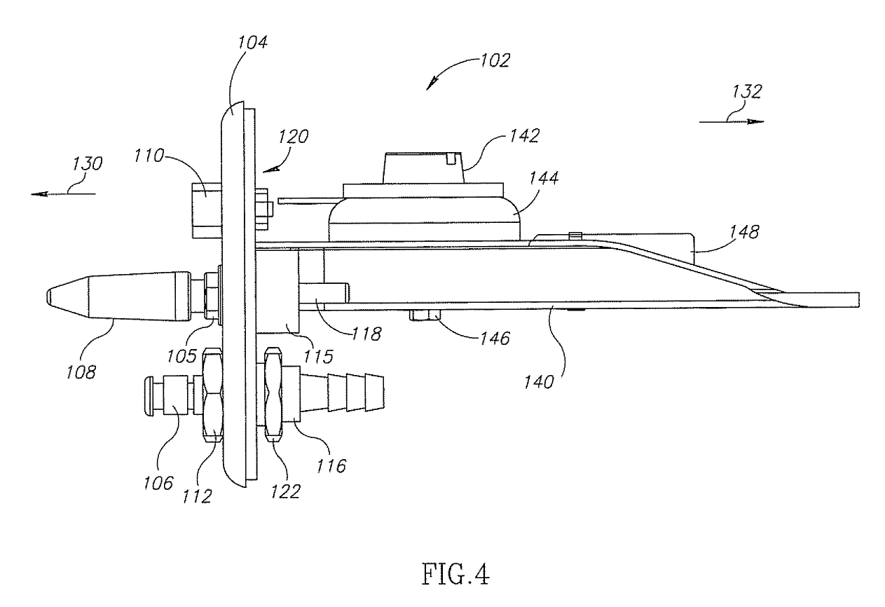

Reference is now made to FIG. 4, which illustrates a close-up internal side view of a connection component of a transducer according to some embodiments. As shown in FIG. 4, the body cover (not shown) of the connection component, 102, has been removed, so that the internal parts located within the hollow part of the connection component body may be observed. Starting from the left side of FIG. 4, connectors, such as connectors 106, 108 and 110, mounted on the external side (generator side, side 130) of cover plate 104 are shown. The connectors may be mounted to the cover plate by various ways, such as for example, pressure, nuts, bolts and the like. For example, connector 106 may be mounted to the external side of cover plate 104 by the use of nut 112. For example, connector 108 may be mounted to the external side of cover plate 104, by the use of a nut such as nut 105. On the other side of cover plate 104, facing towards the internal part of the connection component 102 (end 132), several connectors/fittings may be identified. For example, connector/fitting 116, connector/fitting 118 and connector/fitting 120 may be identified. The connectors/fittings on the internal side of the cover plate, such as cover plate 104, may be mounted to the cover plate by various ways, such as bolts, nuts, pressure and the like. For example, connector/fitting 116 may be mounted by nut 122 and connector/fitting 118 may be mounted by nut 115. The connectors/fittings on the internal side of cover plate 104 may physically and/or functionally interact with the connectors on the external side of cover plate 104. The connectors on the internal side of cover plate 104 may form an integral connector that spans the cover plate from side to side, or may be composed of at least two separate connectors that interact (physically and functionally) through the cover plate. In either case, opposing ends of the connectors face opposing sides of the cover plate. For example, connectors 106 and 116 may form one integral connector that may span the cover plate and may be secured by nuts 112 and 122. The connectors located on the internal side (side 132) of the cover plate may be connected to various pipes, tubes, cables, wires and the like. For example, connector 116 may be connected to fluid pipes. For example, connector 118 may be connected to energy transferring cables. For example, connector 120 may be connected to various wires. In addition, towards the internal side 132 of the connection component, 102, various other parts may be observed. At about the center of the connection component, a carrier plate, such as plate 140 may be observed. Mounted at about the center of carrier plate 140, perpendicularly to the plate is structure 144. Structure 144 may include, a structural element, a functional element and any combination thereof. For example, structure 144 may include a coil that may work in coordination with other components of the transducer, as further detailed herein below. Structure 144 may be secured to carrier plate 140 by a bolt, such as bolt 142 and nuts, such as nut 146. In addition, further to the carrier plate, a card, such as ID card (chip) 148 may be located. ID card 148 may include any kind of information carrying medium that may carry digital and/or analog identifying information regarding the transducer. ID card 148 may include such resources as: information smart card, identification card, security card, memory card and the like, that may include any kind of relevant information regarding the transducer, such as, for example, serial number, specifications, mode of action, length of operation and the like. ID card 148 may further send and receive input to various other components of the transducer and may further send and receive information from external sources, such as a processing unit that may be located in the generator unit. The use of ID card 148 may be to monitor use of the transducer, identify specific information of the transducer, coordinate operation of the transducer, match up operation between the generator unit and the transducer, and the like.

Referencing back to FIG. 2, the vibration component (12) of transducer (2) may be constructed for example of metal, plastic, rigid rubber or any appropriate material. The external casing (housing) of the vibration component may be constructed of one integral structure or may be constructed of several constituents that are interconnected to form the external casing of the vibration component. The vibration component may include at least two main parts that are connected to form the vibration component. The upper part, which is referred to herein as the aiming part (30), is the part of the vibration component that is used to hold and aim the vibration component to a desired location. The lower part of the vibration component, which is referred to herein as the transducing part (32), is the part that may transduce vibration energy and may come in contact (direct or indirect) with a surface to which the energy may be transduced, such as for example, a subject body.

As demonstrated in FIG. 2, the aiming part (30) of the vibration component of the transducer may have a rounded circular disc-like shape. The aiming part may be at least partially hollow and may be externally constructed of two separable parts: a circular disc-like shape which forms a base, and a circular disc-like cover, which may fit onto the base so as to form the at least a partially hollow structure. The top surface of the aiming part may be flat and planar or may be uneven. The top surface may be further coated, covered, adhered with coatings such as rubber, sponge, plastic, paper and the like. The covering of the top surface may further include markings, drawings, sketches, and/or illustrations, such as markings 34A-C, that may be used to assist in aiming and positioning the vibration component. For example, the drawings may include circles (such as 34A-C), curved lines and the like that may correspond to similar markings on surfaces on which the vibration component is to be placed, such as for example, a subject body.

The transducing part (32) of the vibration component (12) of the transducer (2) may be located below the aiming part (30) of the vibration component. The transducing part (32) may be comprised of at least two separable external constituents, such as constituents 36A and 36B in FIG. 2. The separable external constituents (36A-B) may be attached together to form at least a partially hollow casing, which may carry additional internal elements that are described below herein. The separable external constituents (36A-B) may be attached together by various ways, such as by the use of screws, nuts, force, hinges, various fittings or any combination thereof. For example, hole 39 may be employed for placing a screw that may be used to secure constituents 36A and 36 B. The bottom constituent (36A) may have a round shape with protrusions and extensions (such as extension 38A) that may be used to fit with other constituents of the transducer, such as, for example an attachment site for bridging element 14. The upper constituent (36B) may have a generally round shape, with protrusion and extensions (such as, for example, extension 38B) that may be used to fit and/or attach with other constituents of the transducer, such as, for example an attachment site for the bridging element 14. The protrusions and extensions of the bottom constituent and the upper constituent should preferably be complementary, so as to form a closed structure when the constituents are attached. The upper constituent (36B) may, in addition, include two elongated arms that may preferably project upward, such as arms 40A-B illustrated in FIG. 2. Arms, such as arms 40A and 40 B may be an integral part of the upper constituent (36B) and may be used as attachment sites for the aiming part (30) of the vibration component (12). The arms may be length (height) adjustable or may be set to a predetermined length. Attachment between the aiming part (30) and the projected arms (40A-B) of the transducing part (32) may be achieved by various ways, such as by use of pressure, nuts, screws, hinges, or the like. The space thus created between the aiming part and the transducing part may be used for holding, carrying and positioning the transducer to a desired location.

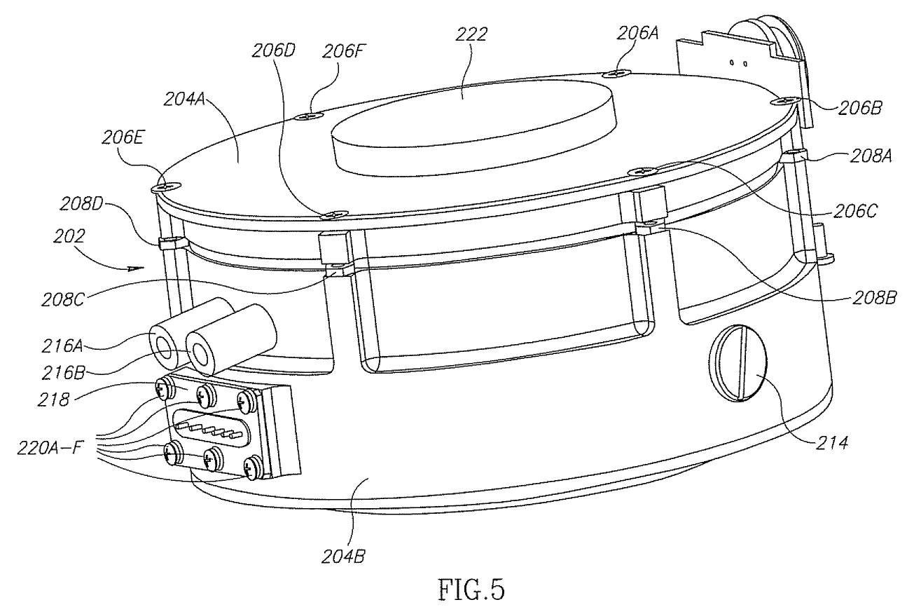

Reference is now made to FIG. 5, which illustrates a perspective view of a transducing part (32 in FIG. 2) wherein the separable external constituents, such as constituents 36A and 36B in FIG. 2 have been removed for clarification. As shown in FIG. 5, within the encasing of the transducing part may be located a component that is referred to herein as a transducing box, 200, which may include the components and elements that may be used to produce vibration energy, such as ultrasound energy, and deliver the energy to a chosen target. Transducing box 200 may include several structural and functional elements, as detailed herein. For example, transducing box 200 may include an outer casing (housing), such as casing (housing) 202 in FIG. 5. The outer casing of transducing box 200 may be constructed of various materials such as plastic, rubber, metal and the like and may have any desirable shape, such as circular, rectangular and the like that may fit in size and function. Outer casing, 202, may preferably be constructed of metal, and may have a circular shape. The outer casing, 202, may form a compartment, with at least a partially hollow interior which may include various other components of transducing box 200, as detailed below. Outer casing 202 may be constructed as one integral part or may be constructed of at least two parts that may be attached in various ways so as to form at least a partially closed compartment. According to some embodiments, outer casing 202 of transducing box 200 may include two parts: a lid, such as lid 204A and a case, such as case 204B. Case 204B may have a round opening at its bottom face, as further detailed below herein. The lid and case of outer casing 202 may be attached and secured one to the other by various ways, such as for example, by use of nuts, screws, blots, force, hinges, fittings and the like. For example, as shown in FIG. 5, lid 204A and case 204B may be attached and secured to each other by the use of screws and nuts, such as screws 206 A-F, and nuts 208 A-D (nut corresponding to screws 206A and 206F are not visible in this figure), that may be fitted onto their respective housings on lid 204A and case 204B. Housings for nuts and screws may include, for example, housings on the lid and housings on the case and may preferably be an integral part of the lid and case, respectively. The location of the housings on lid and case may be such so that each housing on the case may have a corresponding housing on the lid and alignment of the corresponding housings on lid and case would ensure proper attachment of lid and case. Outer casing (such as 202), may further include additional fittings, such as holes, perforations and/or slots that may be fitted to accommodate additional parts of the transducing box. For example, outer casing 202 may include a hole or a fitting for a screw, such as screw 214, that may be used for securing and fitting of elements located within the casing. Screw 214 may further be used as a cap that may be removed so as to allow at least partially filling the interior portion of transducing box 200 with fluid(s). Outer casing 202 may further include, for example, fittings for various connectors, pipes, cables and the like that may be functionally and/or structurally connected to elements located within the outer casing 202 of transducing box 200. For example, holes for connectors such as connectors 216A and 216B may be located in the outer casing, preferably at the case part (204B) of the outer casing. Connectors, such as connectors 216A and 216B may be used to connect to pipes, wires, cables and the like. For examples, connectors 216A and 216B may be used to connect to fluid pipes and may be used for the transfer of fluids (such as gaseous fluids, and/or fluids at their fluid state of aggregation, as for example, water). Connectors such as connectors 216A and 216 B may transverse the outer casing and may be associated with elements located within the casing of transducing box, 200. Outer casing 202 may include fittings for connectors such as connectors 218, that may be used to connect to cables, wires and the like and may be used for the transfer of energy and/or information from sources that are external to the transducing box to elements that are located in the transducing box. Connectors, such as connectors 218 may be functionally and or physically attached to the transducing box. Connectors, such as connectors 218, may transverse the outer casing 202 of transducing box and may be physically and/or functionally connected to and/or associated with elements located within the outer casing of transducing box 200. Connectors, such as connectors 218 may be attached to the outer casing, preferably to the case part (such as case 204B) by various ways, such as for example, by fitting to a hole or perforation in the outer casing, by the use of screws to secure the connectors to their location on the outer casing, by use of force, hinges, nuts, bolts and the like. For example, connectors 218 may be secured to the outer casing by the use of screws, such as screws 220A-F. Location of the fittings for connectors, such as connectors 216A-B and 218 may preferably be on the case part of outer casing 202. Fittings for connectors and connectors such as connectors 216A-B and 218 may preferably be located such that they face the bridging element (such as bridging element 14 in FIG. 2) and may further be located within the extensions 38A-B in FIG. 2 that may form a fitting/attachment site for elements, such as bridging element 14 in FIG. 2. Top surface of lid 204A may be smooth or may include protrusions and/or extensions and may further be at least partially covered/coated with additional elements. The additional elements may include any coating, adhesives, stickers and the like that may have a structural and/or functional supporting elements. The elements that may be attached to lid 204A may be an integral part of lid 204A or may be attached in permanently or reversibly to lid 204A. For example, onto top face of lid 204A, a supporting element, such as element 222 may be attached. Supporting element 222 may be used, for example, to allow the most preferable fitting of transducing box, 202, within the transducing part (32 in FIG. 2). Moreover, supporting element 222, may be used to allow stabilization of transducing box, 202, within the transducing part (32 in FIG. 2). Supporting element 222 may include any shape, size and form and may include one or more elements that may be identical or different in size shape, form and location on lid 204A. For example, supporting element 222 may be constructed of plastic, rubber, sponge, metal, wood, stone and the like and may range in consistency from hard (stiff) to soft. Supporting element 222 may have any shape, such as round, rectangle, triangle, amorphous, and may vary in size. According to some exemplary embodiments, supporting element 222 may be constructed of tough rubber, have a disc-like shape, with a surface area that is about one third of the surface area of top face of lid 204A, be placed at around the center of lid 204A and adhered to place. Preferably, a depression that may fit at least part of supporting element 222 may be located within upper constituent (36B in FIG. 2) of the encasing of transducing part (such as transducing part 32 in FIG. 2).

Reference is now made to FIG. 6, which illustrates a perspective view of the internal parts of transducing box 200 (FIG. 5), according to some embodiments. For clarification, outer casing 202 (FIG. 5) has been removed for clarification, thus revealing internal constituents of transducing box 200. As shown in FIG. 6, a base, such as base (302) may be observed. Base 302 may be substantially round and have a ring like shape, wherein the center of the base is hollow. Base 302 may be constructed of metal, plastic and the like and may be attached by various means, such as bolts, nuts and/or pressure to the outer casing of the transducing box. Preferably, base 302 may be attached to the case part (204B, FIG. 5) of the outer casing of the transducing box. Attachment and alignment of base 302 and case 204B (FIG. 5) may be performed such that the hollow region of base 302 may at least partially fit with the round opening at the bottom face of case 204B (FIG. 5). At the upper face of base 302, situated on the circumference of base 302, clamps, such as clamps 304A, 304 B and 304 C may be observed. Clamps 304A-C may be identical in size and shape and may be constructed of metal, rubber, plastic and the like. Clamps 304A-C may be located at equal distance from each other, along the circumference of base 302. Clamps 304A-C may have a substantially L shape-like structure, with the long arm resting on base 302, and the short arm protruding upwards. Clamps 304A-C may be permanently or reversibly attached to base 302, such as by use of bolts, nuts, gluing, adhering, magnetic attraction, and the like. Clamps 304A-C may be used as carrying parts for transducing element 306. Clamps 304A-C may further be used to adjust the height at which transducing element 306 may be placed above base 302. The use of clamps 304A-C may allow the transducing element to be substantially contact-free from other components of the transducer. According to some embodiments, transducing element 306 may produce therapeutic acoustic energy. Transducing element 306 may include, for example, a piezoelectric element that may be used to produce acoustic waves in response to electrical energy stimulation. The shape, size, thickness, composition and spatial location of the transducing element may be adjusted so as to produce a requested acoustic energy. According to some embodiments, and as demonstrated in FIG. 6, transducing element (such as, for example, transducing element 306) may have a substantially dome-like structure, with a round hole (such as hole 311) substantially at the center of the top of the outer (arched) surface. The transducing element (306) may have substantially smooth surfaces (external arched surface and the internal concaved surface), and the thickness of the element may vary, for example, in the range of 0.1 mm to 100 mm. For example, the thickness of the transducing element may be in the range of 2 mm to 10 mm. Transducing element 306 may be constructed of various components and formulations that may include such materials as metal, ceramics (PZT), and the like. The dome-like shape of transducing element 306 may allow and aid in focusing the acoustic energy produced by the transducing element. Plate 308, located on the circumference of the upper (arched) surface of transducing element 306, may be used as an attachment site for wires, such as electrical wires that may be used to provide electrical energy to transducing element 306. Electrical wires may be attached to plate 308, for example, by welding, soldering and the like. As a result of the electrical energy provided to the transducing element, the element may vibrate and as a result produce acoustic waves and hence acoustic energy. The electrical energy may be provided continuously and a continuous acoustic wave may be produced. According to some embodiments, electrical power provided to the transducing element may be provided in pulses/nodes and the resulting vibration energy produced by the vibration element may be provided in bursts. The electrical power may be in the range of, for example, 1-1000 W. Electrical power provided to the transducing element may be in the range of, for example, 1-750 W. Electrical power provided to the transducing element may be in the range of for example, 1-500 W. Electrical power provided to the transducing element may be in the range of for example, 1-300 W. Electrical power provided to the transducing element may be in the range of, for example, 1-150 W. Electrical power provided to the transducing element may be in the range of, for example, 1-100 W. The transducing element may vibrate at a resonance frequency in the range of about 1 to 1200 kHz. The transducing element may vibrate at a resonance frequency in the range of about 1 to 1000 kHz. The transducing element may vibrate at a resonance frequency in the range of about 1 to 800 kHz. The transducing element may vibrate at a resonance frequency in the range of about 1 to 600 kHz. The transducing element may vibrate at a resonance frequency in the range of about 1 to 400 kHz. The transducing element may vibrate at a resonance frequency in the range of about 1 to 250 kHz. The transducing element may vibrate at a resonance frequency in the range of about 1 to 200 kHz. The transducing element may vibrate at a resonance frequency in the range of about 1 to 150 kHz. The transducing element may vibrate at a resonance frequency in the range of about 1 to 100 kHz. The transducing element may vibrate at a resonance frequency in the range of about 1 to 50 kHz. According to further embodiments, the capacitance of the transducing element may be in the range of, for example, about 0.1 to 15 nF. The capacitance of the transducing element may be in the range of, for example, about 0.1 to 12 nF. The capacitance of the transducing element may be in the range of, for example, about 0.1 to 10 nF. The capacitance of the transducing element may be in the range of, for example, about 0.1 to 9 nF. The capacitance of the transducing element may be in the range of, for example, about 0.1 to 8 nF. The capacitance of the transducing element may be in the range of, for example, about 0.1 to 7.5 nF. The capacitance of the transducing element may be in the range of, for example, about 0.1 to 5 nF. The capacitance of the transducing element may be in the range of, for example, about 0.1 to 2.5 nF. According the other embodiments, the focal diameter of the transducing element, which is the diameter of the region to which the acoustic energy may be focused, may be in the range of, for example, about 0.5 to 20 mm. The focal diameter of the transducing element may be in the range of, for example, about 0.5 to 15 mm. The focal diameter of the transducing element may be in the range of, for example, about 0.5 to 12 mm. The focal diameter of the transducing element may be in the range of, for example, about 0.5 to 10 mm. The focal diameter of the transducing element may be in the range of, for example, about 0.5 to 9 mm. The focal diameter of the transducing element may be in the range of, for example, about 0.5 to 8 mm. The focal diameter of the transducing element may be in the range of, for example, about 0.5 to 7 mm. The focal diameter of the transducing element may be in the range of, for example, about 0.5 to 5 mm. The focal diameter of the transducing element may be in the range of, for example, about 0.5 to 3 mm. The focal diameter of the transducing element may be in the range of, for example, about 0.5 to 2 mm. According to further embodiments, the focal length of the acoustic energy produced by the transducing element may be in the range of, for example, about 1 to 50 mm. The focal length may be in the range of, for example, about 1 to 40 mm. The focal length may be in the range of, for example, about 1 to 35 mm. The focal length may be in the range of, for example, about 1 to 30 mm. The focal length may be in the range of, for example, about 1 to 25 mm. The focal length may be in the range of, for example, about 1 to 20 mm. The focal length may be in the range of, for example, about 1 to 15 mm. The focal length may be in the range of, for example, about 1 to 10 mm. The focal length may be in the range of, for example, about 1 to 5 mm. The focal length may be in the range of, for example, about 1 to 2 mm. According to some embodiments, the focal distance of the focused acoustic energy produced by the transducing element may be measured relative to the working surface, which is the surface to which the energy may be transduced (for example, a subject skin). Thus, the focal distance from the working surface may be in the range of, for example, 1 to 30 mm. The focal distance from the working surface may be in the range of, for example, 1 to 25 mm. The focal distance from the working surface may be in the range of, for example, 1 to 20 mm. The focal distance from the working surface may be in the range of, for example, 1 to 15 mm. The focal distance from the working surface may be in the range of, for example, 1 to 10 mm. The focal distance from the working surface may be in the range of, for example, 1 to 5 mm. The focal distance from the working surface may be in the range of, for example, 1 to 2.5 mm. Acoustic efficiency (as measured in AFB (Acoustic Force Balance system)) of the transducing element may be in the range of, for example, about 1-150 mgr/W. Acoustic efficiency (AFB) of the transducing element may be in the range of, for example, about 10-100 mgr/W. Acoustic efficiency (AFB) of the transducing element may be in the range of, for example, about 15-75 mgr/W. Acoustic efficiency (AFB) of the transducing element may be in the range of, for example, about 20-60 mgr/W. Acoustic efficiency (AFB) of the transducing element may be in the range of, for example, about 25-55 mgr/W. Acoustic efficiency (AFB) of the transducing element may be in the range of, for example, about 29-50 mgr/W. The peak pressure of the transducing element, as may be measured at 1 W of electric power per burst (pulse) may be, for example, in the range of 1 to 800 kPa. The peak pressure of the transducing element, as may be measured at 1 W of electric power per burst (pulse) may be, for example, in the range of 1 to 700 kPa. The peak pressure of the transducing element, as may be measured at 1 W of electric power per burst (pulse) may be, for example, in the range of 1 to 600 kPa. The peak pressure of the transducing element, as may be measured at 1 W of electric power per burst (pulse) may be, for example, in the range of 100 to 800 kPa. The peak pressure of the transducing element, as may be measured at 1 W of electric power per burst (pulse) may be, for example, in the range of 200 to 700 kPa. The peak pressure of the transducing element, as may be measured at 1 W of electric power per burst (pulse) may be, for example, in the range of 300 to 600 kPa. According to some embodiments, the acoustic force provided by each pulse of the transducing element may be controlled by the user. The acoustic force provided by each pulse of the transducing element may be in the range, of for example, about 1-20 gr. The acoustic force provided by each pulse of the transducing element may be in the range, of for example, about 2-15 gr. The acoustic force provided by each pulse of the transducing element may be in the range, of for example, about 4-12 gr. The acoustic force provided by each pulse of the transducing element may be in the range, of for example, about 5-10 gr. The acoustic force provided by each pulse of the transducing element may be in the range, of for example, about 6-8 gr.

According to some embodiments, and as further illustrated in FIG. 6, an additional vibration element, 310, may be observed. Vibration element 310 that is also referred to herein as A-mode, may be situated coaxially at a location that corresponds to the hole at the center of the top of the outer (arched) surface of transducing element 306. A-mode may protrude upwards from a hole (such as hole 311) in transducing element 306. A-mode may include, according to some embodiments, a piezoelectric element that may vibrate and produce acoustic energy in response to electrical stimulation. A-mode may be constructed of various compositions, such as, for example, metal, ceramics and the like and may have any desirable shape, such as amorphous shape, flat, round, rectangular and the like. The energy produced by A-mode may be different than the energy produced by the transducing element 306. A-mode may be used as a monitoring aid (an acoustic probe), which may be used to monitor various parameters during operation of the transducer (2 in FIG. 2), such as, for example, contact with working surface, distance from working surface, and the like. According to some embodiments, A-mode may operate in an echo-mode, wherein acoustic waves are being sent by the A-mode and the feedback signals are being monitored. For example, if no feedback signals are being detected, this may indicate that a sufficient contact between the transducer and the working surface has been achieved. According to some embodiments, the capacitance of the A-mode may be in the range of, for example, about 50 to 2000 pF. The capacitance of the A-mode may be in the range of, for example, about 50 to 1500 pF. The capacitance of the A-mode may be in the range of, for example, about 50 to 1000 pF. The capacitance of the A-mode may be in the range of, for example, about 50 to 800 pF. The capacitance of the A-mode may be in the range of, for example, about 50 to 600 pF. The capacitance of the A-mode may be in the range of, for example, about 50 to 400 pF. The capacitance of the A-mode may be in the range of, for example, about 50 to 200 pF. The capacitance of the A-mode may be in the range of, for example, about 50 to 100 pF. According to further embodiments, the frequency of the A-mode may be in the range of, for example, about 0.5 to 5 Mhz. The frequency of the A-mode may be in the range of, for example, about 0.5 to 4.5 Mhz. The frequency of the A-mode may be in the range of, for example, about 0.5 to 4 Mhz. The frequency of the A-mode may be in the range of, for example, about 0.5 to 3.5 Mhz. The frequency of the A-mode may be in the range of, for example, about 0.5 to 3 Mhz. The frequency of the A-mode may be in the range of, for example, about 0.5 to 2.75 Mhz. The frequency of the A-mode may be in the range of, for example, about 0.5 to 2.5 Mhz. The frequency of the A-mode may be in the range of, for example, about 0.5 to 2.25 Mhz. The frequency of the A-mode may be in the range of, for example, about 0.5 to 2 Mhz. The frequency of the A-mode may be in the range of, for example, about 0.5 to 1.5 Mhz. The frequency of the A-mode may be in the range of, for example, about 0.5 to 1 Mhz. The frequency of the A-mode may be in the range of, for example, about 0.5 to 0.75 Mhz.

As further illustrated in FIG. 6, below the concaved region of transducing element 306, an acoustic coupling interface may be located (312). The acoustic coupling interface may include, for example, a membrane, which may have a round flat bottom (312A), whose diameter may be at least as wide as the diameter of the hollow part of base of transducing element 306; and an upper part whose shape may fit into the concaved region of transducing element 306. The membrane may be constructed of rubber, plastic, silicon, polyurethane and the like and may have desirable acoustic characteristics that may correspond to the acoustic energy produced by the transducing element and the A-mode. A description of an acoustic coupling interface, such as a membrane is detailed herein bellow.

Reference is now made to FIG. 7, which illustrates a perspective view of a cross section of a transducing part, according to some embodiments. The cross section which is illustrated in FIG. 7 is taken along the X-Y plane of the outer encasing of transducing part of FIG. 5. For clarification purposes, numbering of corresponding parts in FIG. 5 and FIG. 7 may be similar. Likewise, numbering of corresponding parts in FIG. 6 and FIG. 7 may be similar. For clarification purposes, lid 204A (FIG. 5) is omitted from FIG. 7. Outer casing 204B has been described in detail in FIG. 5. Briefly, on the outer side of outer casing 204B, housings 608A-B (FIG. 7) may be identified that may house nuts and bolts, such as nuts and bolts 208A of FIG. 5. Housings 606A-B in FIG. 7 may be used as housing for bolts, such as any of bolts 206A-F of FIG. 5. Housing 618 in FIG. 7, may be used as housing for connectors, such as connectors 218 of FIG. 5. Holes 620A-D may be used as housing for any of bolts 220A-F of FIG. 5. Additionally, hole 616 may be observed at the side of outer casing 204B. Hole 616 may be used as housing for connectors, such as any of the connectors 216A-B of FIG. 5. Through connectors 216A-B various substances, such as fluids may enter and exit the transducing box. At the internal side of outer casing 204B several parts, elements and constituents may be observed. Acoustic coupling interface 312 may be observed at lower part of outer casing 204B. Base 312A of the acoustic coupling interface may fit into the round opening at the bottom of outer casing 204B and may protrude out of the outer casing. Above base 312A, the acoustic coupling interface may include a circular shaped rim (312B), whose diameter is larger than the diameter of base 312A. Along the outer circumference of the bottom part of rim 312B, slots and grooves (312C) may be observed. Slots and grooves 312C may be used to ensure proper alignment and placement of acoustic coupling interface 312, onto bottom region of outer casing 204B. The upper part (312D) of the acoustic coupling interface may have a dome-like arched shape. On top of rim 312B of acoustic coupling interface 312, base 302 may be placed. Base 302 has been described above herein with reference to FIG. 6. Base 302 may be secured to the bottom region of outer casing 204B by use of bolts, nuts, glue, pressure, and the like and may indirectly be used to secure the acoustic coupling interface to its place. Securing of base 302 to outer casing 204B while the acoustic coupling interface is situated may clamp the acoustic coupling interface and thus secure the acoustic coupling interface in its place and prevent movement of the acoustic coupling interface. Above acoustic coupling interface 312, is located a transducing element, 306. Transducing element 306 may be situated on three clamps (304 A-C illustrated in FIG. 6), which permit the transducing element to be substantially in the air, which allows it to vibrate freely upon electrical stimulation. The use of clamps may further allow for a distance to form between the acoustic coupling interface and the transducing element. Thus, no direct contact is established between the transducing element and the acoustic coupling interface, and in the space that forms between the transducing element and the acoustic coupling interface acoustic waves and energy may pass. At the top center region of the arched side of the transducing element, a hole may be observed. Vibration element 310 (A-mode) may be situated coaxially at a location that corresponds to the hole at the center of the top of the outer (arched) surface of transducing element 306, A-mode may protrude upwards from the hole in transducing element 306. Additionally, in close proximity to the A-mode, at the region of the center hole at the top of the outer (arched) surface of transducing element 306, is located a thermometer, 610. For example, thermometer 610 may include a EC95F103W thermal sensor (by Thermometrics Inc.). Thermometer 610 may include any kind of thermometer that may be used to measure temperature. For example, thermometer 610 may include a thermo-resistor thermometer. Thermometer 610 may be used to measure temperature of the environment within the transducing box and may be used to monitor operation of the transducing box. Located above the transducing element 306, a reflector plate 612 is located. Reflector plate 612 may be an integral part of outer casing 204B or may be a separable reflector plate that is assembled above the transducing elements. Reflector plate 612 may include a flat plate, a concaved plate or any combination thereof at a thickness of, for example, in the range of between 2 mm to 30 mm. Reflector plate 612 may further include additional lamellar folding(s) and may further include a series of pyramidal-like structures. Reflector plate 612 may be comprised of metal, aluminum, or any other absorbing material, such as, for example, Aptflex F28, Aptflex F48 (both by Precision Acoustics LTD), and the like, and may be used for several purposes. For example, reflector plate 612 may be used to separate the inner volume of the transducing box into at least two compartments: an upper compartment and a lower compartment within the transducing box. The two compartments thus formed may be separable and the volume in one compartment may not mix with the volume of the second compartment. For example, an upper compartment, 614, and a lower compartment 615, may be formed. Upper compartment 614 may be filled with fluids, such as for example water. The fluid may enter and exit the upper compartments via pipes that may connect to the upper compartments by connectors, such as connectors 216A-B (illustrated in FIG. 5) situated at, for example, hole 616. The fluid in the upper compartment may circulate throughout the volume of the upper compartment. Circulation of the fluid in the upper compartment may be used, for example, as a way to lower the temperature in the transducing box. By use of circulating, cold fluid may enter the upper compartment, and thorough circulation in the volume of the upper compartment may absorb heat and leave the upper compartment as a heated fluid. The heat that may be dissipated in this manner may be heat that originates in the lower compartments and may at least partially be transferred to the upper compartment by the reflector plate. Thus, the reflector plate may aid in cooling of the lower compartment and be used as a heat-sink. The lower compartment may be filled with a fluid that may be different than the fluid in the upper compartment. For example, the lower compartment may be filled with oil, such as, for example, paraffin oil. The lower compartment may be filled, for example, after the transducing box has been assembled. The lower compartment may be filled, through, for example, screw (cap) 214 in FIG. 5. The fluid in the lower compartment may fill the spaces and gaps between the various parts and elements situated in the lower compartment, such as, for example, the space between the transducing element and the acoustic coupling interface. The fluid in the lower compartment may need not be circulated. This may mean that the lower compartment may need only be filled once, and the fluid inserted need not be removed. The fluid in the lower compartments may be used, for example, to improve the transfer of acoustic energy within the transducing box. For example, the fluid in the lower compartment may be used to improve acoustic energy transfer from the transducing element to the acoustic coupling interface. Reflector plate 612 may further be used as a wave reflector. For example, reflector plate 612 may be used to reflect (return) acoustic waves that may be emitted from the transducing element. The reflected waves may include, for example, acoustic waves that have been emitted towards the upper compartment and not towards the acoustic coupling interface. The reflector may further include, for example, a pyramidal backscattered design. The pyramidal backscattered design may include a series of aluminum backscattered pyramid-like structures, with a base dimension in the range of 1 to 10 mm. Such a design may aid in scattering the reverse signals (such as wave signals) that are transmitted from the transducing element. The reflector may further include a layer of absorbing material (such as, for example, Aptflex F28 or Aptflex F48 by Precision Acoustics LTD). The layer of absorbing material may have a thickness of, for example, 5 mm to 20 mm and may have any shape, such as a flat surface, concaved surface and the like.

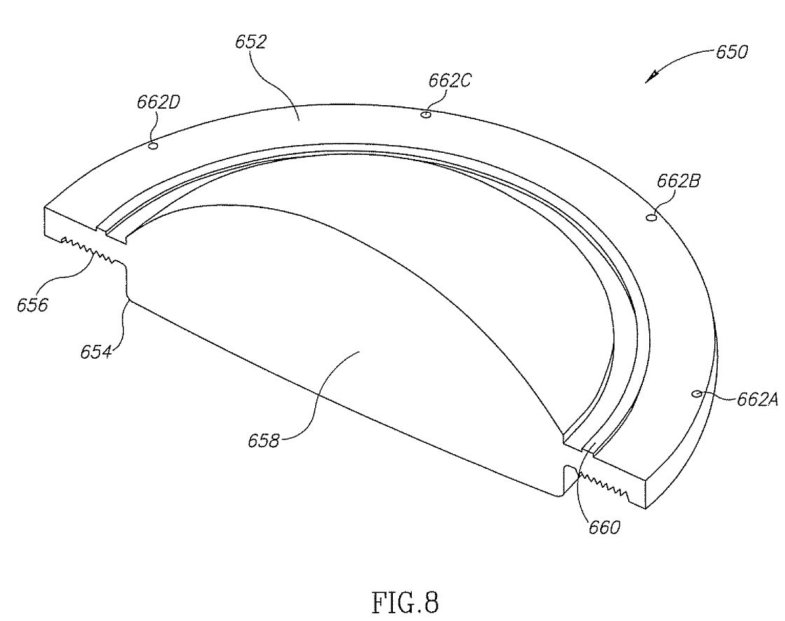

Reference is now made to FIG. 8, which illustrates a perspective midway cross-section view of an acoustic coupling interface, according to some embodiments. The acoustic coupling interface may include a membrane, such as membrane 650 illustrated in FIG. 8A. Membrane 650 may be used to transfer the focused acoustic energy generated by the transducing element to a working surface (that may include, for example, human skin, human soft tissues, and the like). Membrane 650 may be comprised of various materials, such as, for example, rubber, plastic, silicon, polyurethane, and the like. Membrane 650 may be comprised of a biocompatible material. For example, membrane 650 may be comprised of a mixture of two polymers or a bi-component polymer. For example, membrane 650 may be comprised of a mixture of soft polyurethane composition TGS 3740 and JG 5803 (Purchased from Baule, France). The composition of membrane 650 may be correlated to the acoustic energy that may be transferred through the membrane. Membrane 650 may have acoustic properties, such as acoustic impedance similar to that of, for example, soft mammalian tissues to which the membrane may transfer the acoustic energy. Membrane 650 may be comprised as one continuous body, or may be comprised of various parts that may be joined together. Preferably, membrane 650 may be uniformly mixed, such that energy transfer through the membrane is uniform and not deviated and/or absorbed by other objects in the membrane composition, such as, for example, air bubbles. For example, membrane 650 may be prepared in a mold. The at-least partially liquid/non hardened composition of the membrane may be poured into a mold which has a desired shape. Upon polymerization/hardening of the membrane (for example, by a chemical reaction, heating, and the like), the shaped membrane may be released from the mold and ready to be assembled into the transducer. In addition, a mold release substance may be used, that may aid in releasing the shaped membrane from the mold. The mold release composition may include, for example, a release linear that may be comprised of various non-stick substances, such as silicon. Membrane 650 may have a substantially round circular shape, with several distinct regions. The bottom region (654) of membrane 650 is the region that faces the working surface and may come in contact (direct or indirect) with the working surface. The bottom region may have a filled-circular shape with a substantially round circumference. The bottom face of region 654 may be a substantially smooth surface. Above bottom region 654, at a distance, which is about equal to the height of bottom region 654, rim 652 may be observed. Rim 652 may have a larger diameter than bottom region 654 and thus may extend sideways as compared to the bottom region of the membrane. Rim 652 may have a substantially round circular ring-like shape. Rim 652 may have two faces: a bottom face that faces the working surface and a top face that faces the transducing element. At the bottom face of rim 652, grooves (ridges), such as grooves 656 may be observed. Grooves 656 may be located at the bottom face of rim 652 and cover about ⅔ of the width of the lip, starting from the internal circumference of rim 652. Grooves such as grooves 656 may be used for the correct alignment and assembly of the membrane to its location in the transducing box. The grooves may further prevent distortion of the membrane that may be caused by pressure on the membrane parts when assembled in the transducing part. The bottom face of rim 652 may be placed, for example, on encasing 204B of FIG. 7. On the top face of rim 652, an elevated track, 660, may be observed. Elevated track 660 may be located at close proximity to the inner circumference of rim 652 (about ⅕ of the width of rim 652). Elevated track 660 may be have a thickness that is about ⅙ as the thickness of rim 652. Elevated track 660 may be used for the correct alignment and assembly of membrane 650 to its location in the transducing box. In addition, in close proximity to the outer circumference of rim 652, pinholes, such as pinholes 662A-D may be identified. Pin holes 662A-D may be used for the securing of membrane 650 to its location in the transducing box, for example by the use of screws, pins and the like. Onto top face of rim 652, base 302 of FIG. 7 may be placed. Alignment of base 302 (FIG. 7) and rim 652 may be enhanced by fitting the groove in base 302 (FIG. 7) to elevated track 660. Furthermore, securing base 302 (FIG. 7) to membrane 650 and encasing 204B (FIG. 7) may be performed by use of screws, nuts, pins and the like that may fit into holes 662A-D and corresponding holes in base 302 (FIG. 7). Extending above rim 652, membrane 650 may acquire an arched, dome-like structure, 658. The lower region of dome 658 (base) may have a diameter that is substantially similar to the diameter of bottom region 654. Moving upwards, the diameter of the dome may be gradually reduced such that an arched dome-like structure is obtained. Dome-like structure 658 may preferably fit into the concaved area formed by the transducing element (306 in FIG. 7). In addition, membrane 650 may include a thermometer that may include any kind of thermometer that may be used to measure temperature. For example, the membrane thermometer may include a thermo-resistor thermometer. For example, the membrane thermometer may include a EC95F103W thermal sensor (Purchased by Thermometrics Inc.). The membrane thermometer may be placed in close proximity to bottom region 654 and may be used to measure temperature at the working surface. Measurement of temperature at the working surface may be used to ensure proper working conditions and as a health-safety measurement. For example, if the temperature measured by the membrane thermometer is higher than 37 degrees Celsius, the transducer may stop functioning, so as to prevent damage to, for example, a subject skin.

According to some embodiments, upon assembling the membrane into its location in the transducing box, the encasing of the box may be tightly closed. Oil, such as paraffin oil may be inserted into the lower compartments of the transducing box (such as compartment 616 in FIG. 7). Oil may be inserted via screw (cap) 214 (FIG. 5). After oil has been filled, the assembled transducing box may be put in a vacuum for any desired length of time. The vacuum treatment may be used to remove excess air bubbles and other gases that may be trapped in the transducing box. This may allow removing any interfering substances that may interfere and/or attenuate acoustic energy produced in the transducing box. Upon vacuum treatment, the transducing box may be assembled in its housing (outer casing), wires and pipes may be connected and the transducer may be ready for operation.

According to some exemplary embodiments, and as described above herein, a membrane (such as membrane 650 in FIG. 8) may be composed primarily of polyurethane. Prolonged use of the transducer on a subject body may result in damage to the membrane. Such damage may include, for example, protrusion at the center of the working surface of the membrane (654 in FIG. 8), which may eventually lead to complete destruction of the membrane. Throughout operation of the transducer, a membrane (such as membrane 650, FIG. 8) may be subjected to various forces and interactions. For example, vibration pulses, such as ultrasound pulses, produced by the transducer may result in the application of physical pressure, generation of excess heat on the membrane, friction forces applied on the membrane, and the like. The membrane damage, such as protrusion at the center of the of outer surface of the membrane may lead to, for example, distortion of the outer surface of the membrane, which may permit the development and/or introduction of air bubbles to form under the membrane (for example, in the inner part of the membrane). Such a result may have several disturbing effects that may severely influence quality and efficiency of use of the transducer. For example, energy may be produced at skin level of the subject, instead of at a subcutaneous level. This may result in unwanted and unnecessary pain sensation of the subject. In addition, at least a partial attenuation of transducing energy, such as, for example, vibration energy may be caused by existence of air bubbles in the membrane. For example, existence of air bubbles in the membrane may result in at least partial attenuation of pulses produced by the transducing element (as described above). Such attenuation of vibration energy may result in a severe reduction in efficiency and in the expected results of using the transducer.

In addition to the forces described above herein that may damage the membrane, interaction between the membrane and various other external surfaces (such as subject skin, tissues, cells, and the like), substrates, substances and/or materials that may be used during operation of the transducer, may also result in damage to the membrane.

During operation of the transducer, an intermediate substance and/or material, referred to herein as an interposer, may be used, so as to provide an intermediary contact surface between the membrane and the object that may receive the transducing energy, such as a subject body. The interposer may be used to increase efficiency of delivery and/or transmittance of the transducing energy to the subject body. The interposer may include any substance and/or material that may possess such qualities that allow it to be used for the appropriate transmittal of for example, vibration energy from the transducer to a subject body. The interposer should preferably have such qualities as impedance at a range that corresponds to the vibration energy transduced by the transducer and the appropriate range to be received by the target, such as a subject body. Such substances and materials may include, for example, gel, oil, lubricant, ointment, lotion, water, thin rubber layer and the like and may be for example, water based, oil based and the like. For example, the interposer may include ultrasound gel (made by, for example, Medi-Pharm, UK). For example, the interposer used may include castor oil. For example, the interposer used may include paraffin oil. Application of the interposer may be performed by, for example, spreading, spraying, laying, pouring or any other appropriate method of application. The interposer may be applied onto the transducer, onto a subject body, or both.

The interposer may interact and/or come in contact with any part of the transducer. For example, the contact and/or interaction between the interposer and the transducer may include the acoustic coupling interface, such as membrane 650 in FIG. 8. Contact between the interposer and the membrane of the transducer may initiate and/or cause and/or catalyze and/or participate in a reaction that may take place between constituents of the transducer (such as, for example, membrane 650, FIG. 8) and the interposer. The reaction between the interposer and the constituents of the transducer may be acute or may be chronic and may result in at least a partial damage to at least one of the constituents of the transducer. The reaction between the interposer and the constituents of the transducer, may include any kind of reaction, such as, for example, a chemical reaction, a mechanical reaction, heating reaction, and any combination thereof. The chemical reaction between the interposer and constituents of the transducer may result in, for example, at least partial of one or more of the followings: deterioration, destruction, melting, labefaction, weakening, breakage, leakage, protrusion, damage or any combination thereof to at least one of the transducer constituents. The chemical reaction between the interposer and constituents of the transducer may be acute, chronic or a combination thereof. The chemical reaction between the interposer and constituents of the transducer may occur after a short contact/interaction time (such as, for example, in the range of minutes to hours) between the interposer and the constituents of the transducer. The chemical reaction between the interposer and constituents of the transducer may occur after an intermediate contact/interaction time (such as, for example, in the range of hours to days) between the interposer and the constituents of the transducer. The chemical reaction between the interposer and constituents of the transducer may occur after a long contact/interaction time (such as, for example, in the range of days to weeks) between the interposer and constituents of the transducer. The chemical reaction between the interposer and constituents of the transducer may occur after a very long contact/interaction time (such as, for example, in the range of weeks to months) between the interposer and constituents of the transducer. The chemical reaction between the interposer and constituents of the transducer may occur after an even longer contact/interaction time (such as, for example, in the range of months to years) between the interposer and constituents of the transducer. The contact/interaction time between the interposer and constituents of the transducer may be achieved upon continuous contact/interaction, or may be achieved upon several, independent interactions, whose time (which need not be identical) may be added up to get a total time measurement of the contact/interaction. As a non-limiting example, for clarification purposes only, a contact/interaction time of 120 minutes (two hours) between the interposer and constituents of the transducer may be achieved after an uninterrupted, continuous interaction time of 120 minutes (two hours), or may be achieved after, for example, four separate, independent interactions, each one at a length of, for example, 30 minutes.

According to some exemplary embodiments, the interposer may include castor oil and it may interact and/or come in contact with, for example, the membrane of the transducer (such as membrane 650, FIG. 8). The use of castor oil as interposer is preferred because of the acoustic impedance that castor oil possesses. The acoustic impedance of castor oil is approximately the same as the acoustic impedance of the polyurethane membrane of the transducer. The similarity in impedance of the castor oil and the polyurethane membrane may allow maximal vibration energy, such as acoustic energy to be transferred from the transducer to a target, such as a subject body. Contact between the castor oil and the membrane of the transducer may initiate and/or cause and/or catalyze and/or participate in a reaction that may take place between the membrane (such as, for example, membrane 650, FIG. 8) and the castor oil. The reaction between the castor oil and the membrane may be acute or may be chronic and may result in at least a partial damage to the membrane. The reaction between the castor oil and the membrane of the transducer may include a chemical reaction. The chemical reaction between the castor oil and the membrane of the transducer may result in, for example, at least partial of one or more of the followings: deterioration, destruction, melting, labefaction, weakening, breakage, leakage, protrusion, damage or any combination thereof to the membrane of the transducer. The chemical reaction between the castor oil and the membrane of the transducer may be acute, chronic or a combination thereof. The chemical reaction between the castor oil and the membrane of the transducer may occur after a short contact/interaction time (such as, for example, in the range of minutes to hours) between the castor oil and the membrane of the transducer. The chemical reaction between the castor oil and the membrane of the transducer may occur after an intermediate contact/interaction time (such as, for example, in the range of hours to days) between the castor oil and the membrane of the transducer. The chemical reaction between the castor oil and the membrane of the transducer may occur after a long contact/interaction time (such as, for example, in the range of days to weeks) between the castor oil and the membrane of the transducer. The chemical reaction between the castor oil and the membrane of the transducer may occur after a very long contact/interaction time (such as, for example, in the range of weeks to months) between the castor oil and the membrane of the transducer. The chemical reaction between the castor oil and the membrane of the transducer may occur after an even longer contact/interaction time (such as, for example, in the range of months to years) between the castor oil and the membrane of the transducer. The contact/interaction time between the castor oil and the membrane of the transducer may be achieved upon continuous contact/interaction, or may be achieved upon several, independent interactions, whose time (which need not be identical) may be added up to get a total time measurement of the contact/interaction. As a non-limiting example, for clarification purposes only, a contact/interaction time of 120 minutes (two hours) between the castor oil and the membrane of the transducer may be achieved after an uninterrupted, continuous interaction time of 120 minutes (two hours), or may be achieved after, for example, four separate, independent interactions, each one at a length of, for example, 30 minutes.

According to some embodiments, in order to inhibit and/or prevent interaction between the interposer and constituents of the transducer, a barrier, such as a protective barrier may be used. The protective barrier may include a physical barrier, a chemical protective barrier, or any combination thereof, and may prevent the physical interaction, and or reaction between the interposer and constituents of the transducer. The protective barrier may include, for example, a chemical protective barrier. The protective barrier may be applied by any method known in the art, such as, for example, but not limited to: coating, spraying, brushing, applying, adhering, placing, and the like. The protective barrier may be permanently applied. The protective barrier may be transiently applied, during the time an interaction between the interposer and constituents of the transducer takes place, such as for example, during use of the transducer. The protective barrier may be preferably applied to the constituents of the transducer that may need such protection. The protective barrier may posses such qualities that it may not interfere with the transmittance, transduction, delivery of energy from the transducer to the target, such as for example, a subject body.

According to some exemplary embodiments, the interposer may include castor oil, and the constituent of transducer that may interact with the castor oil is membrane, such as membrane 650 in FIG. 8. In order to prevent interaction and/or reaction between the castor oil and the membrane, a protective barrier may be used. The protective barrier may include a chemically protective barrier that may inhibit a possible chemical reaction between the castor oil and the polyurethane membrane. The protective barrier may be preferably applied to the membrane of the transducer. The protective barrier may be applied by any method known in the art, such as, for example, but not limited to: coating, spraying, brushing, air brushing, drying onto, applying, adhering, placing, and the like, or any combination thereof. The protective barrier may be permanently applied to the membrane. The protective barrier may be transiently applied to the membrane. Transient application of the protective barrier to the membrane of the transducer may be such that the protective barrier is applied to the membrane a short time before the interaction with the castor oil commences, and stays applied to the membrane at least during the time period of interaction between the castor oil and the membrane, such as for example, during use of the transducer. The protective barrier may posses such qualities so that it may not interfere with the transmittance, transduction, and delivery of energy from the transducer to a target.

According to some embodiments, the protective barrier may be permanently applied to the membrane of the transducer. The protective barrier may be applied by any method known in the art, such as for example, but not limited to: coating, spraying, brushing, air brushing, drying onto, applying, adhering, placing, and the like, or any combination thereof. The protective barrier that may be permanently applied to the membrane may form an integral part of the membrane. For example, the protective barrier may be added to the mixture from which the membrane is prepared. For example, the protective barrier may be molded along with the other constituents of the membrane, such as polyurethane to produce an integral, protectively coated membrane. In another embodiment, the membrane may be molded in regular fashion, assembled into its housing in the transducer and then coated on the outer surface with a protective barrier. The protective barrier may be applied one or more times, to create, for example, one layer or multiple layers of coating.

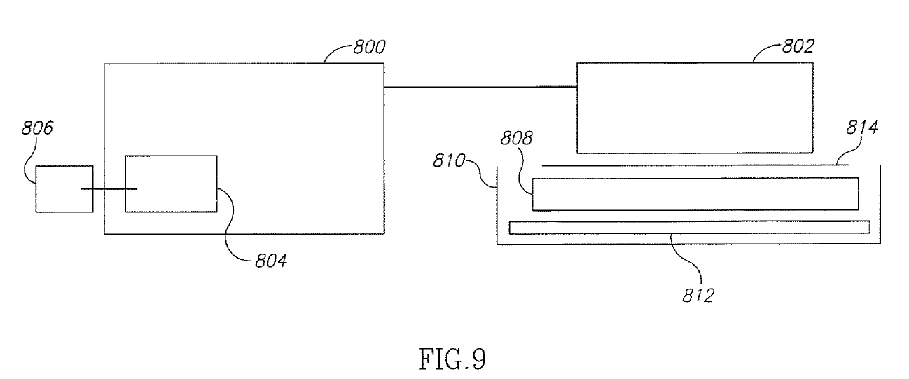

Reference is now made to FIG. 9, which illustrates a simplified schematic drawing of experimental setup that may be used to test transducer related parameters. As shown in FIG. 9, a generator unit 800 may be attached to a transducer, 802. The generator unit may include a pulser (804) that may provide the transducer with pulsed electrical energy that may be used by the transducer to produce acoustic energy. The generator unit may further be connected to Oscilloscope 806 (for example, Textronix, 2 channels). The Oscilloscope may be adapted to detect signals from the A-mode of the transducer. The transducer 802 may be placed over a phantom (808), which may mimic a working surface, such as a mammalian subject. Phantom 808 may include, for example, a polyurethane material (hardness: shore 0, as determined by a shore durometer, dimensions: 19×19×3 cm). Phantom 808 may be placed in a tank 810, and underneath the phantom an absorber (812) may be placed. The absorber may include absorbing material such as Aptflex F48 at 10 mm thickness (purchased from Precision Acoustics Ltd.). The absorbing material may be used to absorb acoustic energy and may further enable measurements of the absorbed energy. On top of Phantom 808, an interposer (814), such as castor oil, may be placed. Upon assembly of the experimental setup, the transducer may be tested. The transducer may be operated under various operating conditions. Operating conditions may include, for example, at frequency of 200 kHz: duty cycle may be in the range of ½ to 1/50 (for example, about 1/10, ½ or 1/30); burst length may be in the range of 5 to 500 (for example, about 100, 300 or 400); treatment time (node time) may be in the range of 0.5-5 seconds (for example, about 1, 2 or 3 seconds); electrical power provided to the transducer may be at the range of 100 to 500 W (for example, 200, 250, 300 or 400 W). Transducer related parameters, such as acoustic properties (hydrophone measurements, AFB measurements) and electrical properties (such as impedance scan) may be measured. The transducer related parameters thus measured may be compared under various experimental conditions, such as for example, measurements at various time points, measurements with or with out protective coatings, and the like.

According to some exemplary embodiments, the protective barrier may be permanently applied to the membrane of the transducer. The coating material comprising the protective barrier may include various substances that may be adapted to serve as a chemically protective barrier and to prevent chemical reaction between castor oil and the polyurethane membrane. Some examples (see example 1) of formulations and substances that may be used as protective coating may include: PO-40 (formulation 19-55-4); coating material may include formulation 19-52-7, coating material may include formulation 19-55-1; coating material may include formulation 19-55-2; coating material may include formulation 19-55-3; coating material may include silicon formulations; coating material may include Parylene formulations, and the like. Mold release material, which may be used to cover the mold template prior to molding the membrane, may include PVA solution. The mold release material may include mold release of formula 19-56-5 (example 1). The mold release material may include silicon formulations, (such as, for example, Silicon RTV 250 Alchemix) at various thickness, such as for example 12 mm and 22 mm. The mold release material may include any combination of mold release materials, such as, for example, silicon based and Polyurethane based formulations. Application of coating material, such as coating materials described above herein, may be performed, for example, by any of the following options:

A. molding a coated membrane, wherein the coating material forms an integral part of the molded membrane. Upon molding the coated membrane it may be assembled in its housing in the transducer. The transducer may then be put in vacuum, as part of the regular assembly procedure. Under this option, the process must be very accurate and may take more time.

B. coating of a membrane after it has already been molded. Under this option, a membrane is molded in regular fashion. The membrane may then be assembled in the transducer housing. The transducer may then be put in vacuum, as part of the routine assembly procedure. After vacuum treatment, the protective coating may be applied to the outer surface of the membrane. Application of the coating to the outer surface of the membrane may be performed, for example, by spraying the outer surface of the membrane with the protective coating. Spraying of the coating onto the surface of the membrane may be performed, for example, by use of air brushes, such as air brushes VL and H type. After application of the coating material is completed, assembly of the transducer may be finished.

C. coating of a membrane after it has already been molded. Under this option, a membrane is molded in regular fashion. The membrane may then be assembled in the transducer housing. The protective coating may then be applied to the outer surface of the membrane. Application of the coating to the outer surface of the membrane may be performed, for example, by spraying the outer surface of the membrane with the protective coating. Spraying of the coating onto the surface of the membrane may be performed, for example, by use of air brushes, such as air brushes VL and H type. After application of the coating material is completed, the transducer may be put in vacuum. After vacuum treatment, assembly of the transducer may be finished. Thickness of the coating may be in the range of about 1 to 20 micron. Examples 2-5 detail the results of experiments of testing the effect of coating materials on transducer parameters during the life span of the transducer. The results in examples 2-5 detail the effects of castor oil application on membranes coated by various coating materials by using some of the coating option as described above.

According to some exemplary embodiments, the protective barrier may be permanently applied to the membrane of the transducer. The coating may be applied by gluing and/or adhering to the outer surface of the membrane. The coating may be supplied, for example, in the form of a sticker, label, peel-off, flexible membrane, and the like, or any combination thereof. The protective barrier may be thin, such as in the range of about 0.05 mm. The protective coating may be glued to the membrane in various ways, such as, for example, by use of UV glue. The protective coating may be comprised of, for example, formulations containing PVC, acrylic BOPP material, formulations containing silicon and the like, and any combination thereof.

According to some embodiments, the protective barrier may include commercially available coating stickers that may be used as a protective barrier between castor oil and the membrane of the transducer. Some non-limiting examples of protective barriers that may be used include PVC stickers (made by Linero Color); stickers made by Avery company, such as, for example, Avery FasCal 400/440 Permanent Scoreback; stickers made by 3M company, such as, for example, models no.: 9793R, 1521, 1520, 1523, 1516, 1526. Testing of the coating stickers as protective barriers may be performed in various ways. According to some examples, testing may be performed to check if a reduction in acoustic/electric properties of transducers is observed during life expectancy testing of coated membranes. Methods of checking acoustic/electrical parameters may include simulation of treatment using a transducer and tracking various transducer related parameters during the lifetime of the transducer. Example 6 details sets of experiments testing the effect of coating materials on transducer parameters during the life span of the transducer. The results demonstrate that the focal distance, focal length and transducer diameter did not change. Measurements of the power (6 dB, 40 dB) at focus and at 1 mm under polyurethane show that the values at the end of the life expectancy of the transducer are, in general, higher than to start with. This may be explained by improvement in the adhesion of the sticker over time and consequently the disappearance of micro bubbles that may form inside the glue that is used to adhere the coating to the membrane.

Application of a protective barrier, such as by gluing, adhering, sticking and the like, may be performed after the membrane has been molded and introduced into the housing of transducer. Application of the coating may be performed in such a way that complete adherence between the coating and the membrane is achieved. Application of the coating may be performed in such a way that a direct and uninterrupted contact is established between the membrane and the coating. The interaction between the coating and the membrane may be such that the entire outer surface area of the membrane is completely covered/coated by the coating material. The completely intimate contact between the outer surface of the membrane and the coating may not allow any other substance and/or material, such as liquid, air and the like to penetrate and/or form in the contact area between the membrane and the coating.

According to some preferred embodiments, the membrane of the transducer may be coated with a protective barrier. The protective barrier may be used to prevent interaction and chemical reaction between the membrane and castor oil, that may be used as an interposer. The protective barrier may include, for example, a commercially available polyolefin film, (such as product number 9793R of 3M Company). The polyolefin film may consist of a clear polyolefin film coated on one side with a pressure sensitive acrylate adhesive. The thickness of the polyolefin film may be in the range of about 30 to 500 microns. The polyolefin coating may be typically 0.05 mm thick. The acrylate adhesive may be, for example 0.03 mm thick. The film may be supplied on a silicon coated release liner that may improve ease of use of the coating. The film may be suitable for use at a wide range of working temperatures, such as in the range of −70 and 100 Celsius degrees. The acrylate adhesive may be applied, according to some embodiments, to the outer surface of the membrane of the transducer, in such a way that the protective polyolefin coating may face the interposer, such as castor oil.

According to some embodiments, the protective barrier that may be used may be heat resistant and sufficiently rigid so as to maintain its shape and form, and further maintain the shape of the working surface of the membrane. The glue, or adhering method used to attach the coating to the membrane should be heat stable so as to withstand various operation conditions of the transducer. The protective barrier may preferably not interfere with the transfer of acoustic energy from the transducing element to the working surface. Furthermore, the protective barrier may preferably not interfere with the transfer of acoustic energy from the A-mode transducer to the working surface. The protective barrier may have a smooth surface, which may lower the friction between the membrane and the working surface. Lowering the friction may increase life expectancy of the membrane and the protective barrier. Furthermore, lowering the friction may improve working conditions. This may mean that the working surface, such as subject skin, is less subject to friction forces, and thus pain and discomfort levels are reduced. In addition, a user that operates the transducer may experience easier operation, as less resistance is observed due to lower friction between the transducer and the working surface.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

### EXAMPLES

### Example 1

### Formulations of Various Compositions Described Above Herein

| | | |
|---|---|---|
| Parts | Material | Supplier |

1. Mold release (19-56-5)

| | | | |
|---|---|---|---|
| 50 | gram | PVA mold release solution | El-Gad, Rival Street |
| | | | Tel-Aviv |
| 50 | gram | 80/20 w/w 96% Ethanol/water | Any lab supplier |
| 36 | mg | Methylene blue dye | Sigma-Aldrich, M4490-7 |
| 0.15 | gram | Byk 348 | Myko Engineering |
| 25 | mg | Sodium Azide | Sigma-Aldrich, S-2002 |

2. Protective coating (19-55-1)

| | | |
|---|---|---|
| 6 | Estane 5701 FIP | Raw-Mat, Mazkeret Bathya |
| 36 | MEK | Lab supplier |
| 18 | Toluene | Lab supplier |
| 20 | THF | Lab supplier |
| 31.8 | cyclohexanone | Lab supplier |
| 0.2 | Byk 307 | Myko Engineering |

3. Protective coating (19-55-2)

| | | |
|---|---|---|
| 6 | Estane 5701 FIP | Raw-Mat, Mazkeret Bathya |
| 30 | MEK | Lab supplier |
| 18 | Toluene | Lab supplier |
| 10 | THF | Lab supplier |
| 48 | cyclohexanone | Lab supplier |
| 0.2 | Byk 307 | Myko Engineering |

4. Protective coating 19-55-3

| | | |
|---|---|---|
| 6 | Estane 5701 FIP | Raw-Mat, Mazkeret Bathya |
| 24 | MEK | Lab supplier |
| 18 | Toluene | Lab supplier |
| 64 | cyclohexanone | Lab supplier |
| 0.2 | Byk 307 | Myko Engineering |

5. Protective coating PO-40, 19-55-4

| | | |
|---|---|---|
| 200 | PO 40.15 adhesive solution | Adhestick, Rosh Haain |
| | (15% solids) | |
| 300 | MIBK | Lab supplier |
| 25 | Butyl glycol | Lab supplier |
| 1.0 | Byk 307 | Myko Engineering |

6. Protective coating 19-52-7

| Gr. | Material |
|---|---|
| 10 | MEK |
| 8 | MIBK |
| 4 | Xylen |
| 1.2 | O.blue in aceton |
| 6 | cyclohexane |
| 5 | Toluene |
| 1.7 | THF |
| 3.5 | Estane 5701 FIP |
| 1.5 | Byk 307 |

### Example 2

### Use of PO-40 as Coating Material of Polyurethane Membrane

Tools and Materials:

### 1. Coating Material: PO-40

2. Mold release material: PVA solution (El-Gad)
3. Air brushes: VL and H type (ARTA)

### 4. Air Compressor

### Description:

The transducer was assembled according to option C, described above herein. Upon assembling the transducer and coating the membrane by spraying, 5 big drops of Castor oil were laid on the membrane. Every few hours one of the drops was removed and visual effect/damage on the membrane that might have been caused by the Castor oil were sought. Results are summarized below:

| Time | Duration [Hours:Min] | Action | Effect/Damage |
|---|---|---|---|
| 15:15 | 0 | Start | No Damage |
| 18:10 | 2:55 | Removing drop # 1 | No Damage |
| 8:15 | 17:00 | Removing drop # 2 | No Damage |
| 16:55 | 25:40 | Removing drop # 3 | No Damage |
| 10:00 | 42:45 | Removing drop # 4 | No Damage |
| 17:45 | 50:25 | Removing drop # 5 | No Damage |

In addition, impedance scans and acoustic output measurements were performed at various time points, and compared between coated and non coated membranes. Results are summarized below:

### Regular Polyurethane Membrane

| | Work | | | Focal | Focal | Focal | Peek | |
|---|---|---|---|---|---|---|---|---|
| | freq. | \|Z\| | Coil | distance | length | diam. | Press | Pacos |
| Processes or Actions or Notes | [kHz] | [Ohm] | [uH] | [mm] | [mm] | [mm] | [kPa] | [W] |
| Impedance Scan in: Vin = 1 V | 178 | 32 | 18.6 | | | | | |
| Acoustic Output @ Z = 0 mm, 30 × 30, | 187 | | | 12.5 | 11.73 | 6.25 | 706 | 1.359 |
| 31pnt | | | | | | | | |
| Acoustic Output @ Z = 1 mm from PU | | | | | | 13.6 | 331 | 1.606 |
| Plane, 50 × 50, 31pnt | | | | | | | | |

### Coated Polyurethane Membrane

| | Work | | | Focal | Focal | Focal | Peek | |
|---|---|---|---|---|---|---|---|---|
| | freq. | \|Z\| | Coil | distance | length | diam. | Press | Pacos |
| Processes or Actions or Notes | [kHz] | [Ohm] | [uH] | [mm] | [mm] | [mm] | [kPa] | [W] |
| Impedance Scan in: Vin = 1 V | 178 | 36.3 | 21.7 | | | | | |
| Acoustic Output @ Z = 0 mm, 30 × 30, | 187 | | | 9.41 | 22.37 | 6.2 | 739 | 1.513 |
| 31pnt | | | | | | | | |
| Acoustic Output @ Z = 1 mm from PU | | | | | | 13.5 | 374 | 1.771 |
| Plane, 50 × 50, 31pnt | | | | | | | | |

### AFB Measurements

| Trans./ | | | | |
|---|---|---|---|---|
| Freq.[KHz] | Vin | Power [W] | Force [gr] | Efficiency [mg/W] |
| No Coating/178 | 80 | 158 | 0.53 | 33.45 |
| With Coating/178 | 80 | 146 | 0.54 | 36.98 |
| No Coating/187 | 80 | 159 | 0.53 | 33.26 |
| With Coating/187 | 80 | 156 | 0.56 | 35.87 |

### Example 3

### Use of Formulation 19-52-7 as Coating Material of Polyurethane Membrane

### Tools and Materials:

### 1. Coating Material: 19-52-7

2. Mold release material: PVA solution (El-Gad)
3. Air brushes: VL and H type (ARTA)

### 4. Air Compressor

### Description:

The transducer was assembled according to option C, described above herein. Upon assembling the transducer and coating the membrane by spraying, 5 big drops of Castor oil were laid on the membrane. Every few hours one of the drops was removed and visual effect/damage on the membrane that might have been caused by the Castor oil were sought. In addition, impedance scans and acoustic output measurements were performed at various time points, and compared between coated and non-coated membranes. Results are summarized below:

### Before Coating

| | Work | | | Focal | Focal | Focal | Peek | |
|---|---|---|---|---|---|---|---|---|
| | freq. | \|Z\| | Coil | distance | length | diam. | Press | Pacos |
| Processes or Actions or Notes | [kHz] | [Ohm] | [uH] | [mm] | [mm] | [mm] | [kPa] | [W] |
| Impedance Scan in: Vin = 1 V | 211 | 34 | 1.33 | | | | | |
| Acoustic Output @ Z = 0 mm, 30 × 30, | 211 | | | 12.3 | 21.1 | 5.5 | 792 | 1.1216 |
| 31pnt | | | | | | | | |
| Acoustic Output @ Z = 1 mm from PU | | | | | | 24.5 | 205 | 1.2404 |
| Plane, 50 × 50, 31pnt | | | | | | | | |

### Coated Polyurethane Membrane

| | Work | | | Focal | Focal | Focal | Peek | |
|---|---|---|---|---|---|---|---|---|
| | freq. | \|Z\| | Coil | distance | length | diam. | Press | Pacos |
| Processes or Actions or Notes | [kHz] | [Ohm] | [uH] | [mm] | [mm] | [mm] | [kPa] | [W] |
| Impedance Scan in: Vin = 1 V | 211 | 32.7 | 1.35 | | | | | |
| Acoustic Output @ Z = 0 mm, 30 × 30, | 211 | | | 11.4 | 21.67 | 5.54 | 767 | 1.0904 |
| 31pnt | | | | | | | | |
| Acoustic Output @ Z = 1 mm from PU | | | | | | 18 | 234 | 1.203 |
| Plane, 50 × 50, 31pnt | | | | | | | | |

### Example 4

### Use of 19-55-3 Material as Coating Material of Polyurethane Membrane

### Tools and Materials:

1. Coating Material: Medical grade material
2. Mold release material: PVA solution (El-Gad) 19-51-5
3. Air brushes: VL and H type (ARTA)

### 4. Air Compressor

### Description:

The transducer was assembled according to option C, described above herein. Upon assembling the transducer and coating the membrane by spraying, 5 big drops of Castor oil were laid on the membrane. Every few hours one of the drops was removed and visual effect/damage on the membrane that might have been caused by the Castor oil were sought. In addition, impedance scans and acoustic output measurements were performed at various time points, and compared between coated and non-coated membranes. Results are summarized below:

### Before Coating

| | Work | | | Focal | Focal | Focal | Peek | |
|---|---|---|---|---|---|---|---|---|
| | freq. | \|Z\| | Coil | distance | length | diam. | Press | Pacos |
| Processes or Actions or Notes | [kHz] | [Ohm] | [uH] | [mm] | [mm] | [mm] | [kPa] | [W] |
| Impedance Scan in: Vin = 1 V | 211 | 34 | 1.33 | | | | | |
| Acoustic Output @ Z = 0 mm, 30 × 30, | 211 | | | 12.3 | 21.1 | 5.5 | 792 | 1.1216 |
| 31pnt | | | | | | | | |
| Acoustic Output @ Z = 1 mm from PU | | | | | | 24.5 | 205 | 1.2404 |
| Plane, 50 × 50, 31pnt | | | | | | | | |

### Coated Polyurethane Membrane

| | Work | | | Focal | Focal | Focal | Peek | |
|---|---|---|---|---|---|---|---|---|
| | freq. | \|Z\| | Coil | distance | length | diam. | Press | Pacos |
| Processes or Actions or Notes | [kHz] | [Ohm] | [uH] | [mm] | [mm] | [mm] | [kPa] | [W] |
| Impedance Scan in: Vin = 1 V | 211 | 34 | 1.33 | | | | | |
| Acoustic Output @ Z = 0 mm, 30 × 30, | 211 | | | 13.2 | 21.7 | 5.85 | 757 | 1.2249 |
| 31pnt | | | | | | | | |
| Acoustic Output @ Z = 1 mm from PU | | | | | | 24.2 | 210 | 1.1181 |
| Plane, 50 × 50, 31pnt | | | | | | | | |

Setup: f.g. + Amp + transformer 50/1 ohm + measurements box (1 ohm)

### Example 5

### Use of 15-55-4 as Coating Material of Polyurethane Membrane

Experiments were performed essentially as described in FIG. 9. Several transducers were tested for number of pulses before damage to membrane was detected.

### List of Transducers

| | Frequency |
|---|---|
| Transducer | [kHz] |
| 1-02-1177L | 200 |
| 1-03-1767L | 186 |

After less than 10000 nodes damage to membrane was observed.

### Example 6

### Use of Sticker 3M9793R Material as Coating of Polyurethane Membrane

Experiments were performed essentially as described in FIG. 9. Several transducers were tested over a period of approximately 2 months at about 7-14 day intervals.

### List of Transducers

| | Frequency |
|---|---|
| Transducer | [kHz] |
| 1-02-0504L | 199 |
| 1-02-0652L | 206 |
| 1-02-0058L | 177 |
| 1-02-0097L | 172 |
| 1-02-0483L | 210 |
| 1-02-0111L | 174 |
| 1-02-0263L | 196 |
| 1-02-1012L | 204 |

### AFB Measurements

| | | # nodes | |
|---|---|---|---|
| Transducer | Sticker | (pulses) | Eff [mg/W] |
| 1-02-0652L | 3M9793R | 0 | 27.04 |
| 1-02-0652L | 3M9793R | 20000 | 27.21 |
| 1-02-0652L | 3M9793R | 35000 | 28.87 |
| 1-02-0652L | 3M9793R | 46000 | 28.71 |
| 1-02-0058L | 3M9793R | 0 | 32.06 |
| 1-02-0058L | 3M9793R | 20000 | 28.7 |
| 1-02-0058L | 3M9793R | 30000 | 30.91 |
| 1-02-0058L | 3M9793R | | |
| 1-02-0097L | 3M9793R | 15000 | 23.8 |
| 1-02-0097L | 3M9793R | 35000 | 25.51 |
| 1-02-0097L | 3M9793R | 43000 | 26.8 |
| 1-02-0504L | 3M1520 | — | 29.98 |
| 1-02-0504L | 3M9793R | 20000 | 27.79 |
| 1-02-0504L | 3M9793R | 40000 | 28.18 |
| 1-02-0483L | no | — | 35.62 |
| 1-02-0483L | 3M9793R | 0 | 31.83 |
| 1-02-0483L | 3M9793R | 12000 | 29.9 |
| 1-02-0483L | 3M9793R | 32000 | 29.88 |
| 1-02-0483L | 3M9793R | | |
| 1-02-0111L | no | — | 32.27 |
| 1-02-0111L | 3M9793R | 0 | 32.05 |
| 1-02-0111L | 3M9793R | 10000 | 31.13 |
| 1-02-0111L | 3M9793R | 30000 | 31.98 |
| 1-02-0111L | 3M9793R | 36000 | 32.11 |
| 1-02-0111L | 3M9793R | | 33.09 |
| 1-02-0263L | no | — | 30.89 |
| 1-02-0263L | 3M9793R | 0 | 28.73 |
| 1-02-0263L | 3M9793R | 15000 | 29.09 |
| 1-02-0263L | 3M 9793R | 42000 | 30.16 |
| 1-02-1012L | no | — | 31.43 |
| 1-02-1012L | 3M9793R | 0 | 29.63 |
| 1-02-1012L | 3M9793R | 23000 | 30.1 |
| 1-02-1012L | 3M9793R | | 28.57 |

### NTR Measurements (Characterization of Acoustic Field)

1 mm under PU

| | | | | | | | | | | | | PP |
|---|---|---|---|---|---|---|---|---|---|---|---|---|
| | | | Focal | Focal | Transd | Power | Power @ | Peak | Power | Power @ | Peak | focus/ |
| | | # nodes | Dist | Length | diameter | @6 dB | 40 dB | Pressure | @6 dB | 40 dB | Pressure | PP |
| Transducer | 9 Sticker | (Pulses | [mm] | [mm] | [mm\hat{ }2] | [mW] | [mW] | [kPa] | [mW] | [mW] | [kPa] | 1 mm |
| 1-02-0504L | 3M9793R | 20000 | 12.8 | 22 | 6.7 * 5.8 | 455 | 1020 | 513 | 526 | 772 | 150 | 3.4 |
| 1-02-0504L | 3M9793R | 40000 | 13 | 21.7 | 5.8 * 5.5 | 468 | 1080 | 538 | 591 | 891 | 175 | 3.1 |
| 1-02-0652L | 3M9793R | 0 | 12.6 | 23.4 | 5.8 * 5.7 | 510 | 1070 | 550 | 754 | 924 | 146 | 3.8 |
| 1-02-0652L | 3M9793R | 20000 | 12.6 | 23.5 | 5.8 * 5.7 | 498 | 1060 | 538 | 721 | 853 | 159 | 3.4 |
| 1-02-0652L | 3M9793R | 35000 | 14.3 | 23.4 | 5.8 * 5.7 | 506 | 1040 | 538 | 700 | 846 | 134 | 4.0 |
| 1-02-0652L | 3M9793R | 46000 | 13.8 | 23.9 | | 490 | 1060 | 538 | 733 | 874 | 146 | 3.7 |
| 1-02-0058L | 3M9793R | 0 | 12.4 | 23.6 | 6.9 * 6.9 | 1070 | 1650 | 595 | 846.2 | 1500 | 257 | 2.3 |
| 1-02-0058L | 3M9793R | 20000 | 12 | 22.7 | 6.5 * 6.6 | 953 | 1600 | 568 | 725 | 1500 | 243 | 2.3 |
| 1-02-0058L | 3M9793R | 30000 | 13 | 23.9 | 6.6 * 6.7 | 1010 | 1600 | 681 | 649 | 1460 | 256 | 2.3 |
| 1-02-0058L | 3M9793R | | 12.8 | 23.05 | 7.1 * 6.9 | 1200 | 1800 | 642 | 1080 | 1730 | 270 | 2.4 |
| 1-02-0097L | 3M9793R | 15000 | 12.3 | 26 | 7.2 * 7.05 | 713 | 1200 | 482 | 696 | 1200 | 234 | 2.1 |
| 1-02-0097L | 3M9793R | 20000 | 13.6 | 24.97 | 6.7 * 6.7 | 722 | 1290 | 496 | 661 | 1160 | 234 | 2.1 |
| 1-02-0097L | 3M9793R | 43000 | 12.6 | 24.2 | 6.9 * 6.8 | 811 | 1400 | 523 | 829 | 1230 | 220 | 2.4 |
| 1-02-0483L | no | 0 | 14.2 | 20 | 5.5 * 5.6 | 591 | 1180 | 629 | 719 | 964 | 169 | 3.7 |
| 1-02-0483L | 3M9793R | 12000 | 13.3 | 19.9 | 5.4 * 5.7 | 680 | 1390 | 701 | 759 | 1090 | 192 | 3.7 |
| 1-02-0483L | 3M9793R | 32000 | 12.5 | 19.9 | 5.3 * 5.7 | 729 | 1410 | 726 | 743 | 1140 | 205 | 3.5 |
| 1-02-0483L | 3M9793R | | 14.2 | 20.8 | 5.6 * 5.8 | 903 | 1650 | 846 | 674 | 1120 | 205 | 4.1 |
| 1-02-0111L | 3M9793R | 0 | 13.8 | 24 | 6.9 * 6.7 | 768 | 1329 | 513 | 1053 | 1600 | 184 | 2.8 |
| 1-02-0111L | 3M9793R | 10000 | 13.6 | 23.6 | 6.8 * 6.7 | 755 | 1350 | 519 | 953 | 1320 | 218 | 2.4 |
| 1-02-0111L | 3M9793R | 30000 | 14 | 25.1 | 6.9 * 6.9 | 953 | 1640 | 574 | 1220 | 1740 | 259 | 2.2 |
| 1-02-0111L | 3M9793R | | 13 | 23.9 | 6.8 * 6.7 | 1040 | 1733 | 588 | 917 | 1910 | 243 | 2.4 |
| 1-02-0263L | 3M9793R | 15000 | 14.7 | 20.44 | 5.9 * 5.9 | 622 | 1091 | 594 | 804 | 957 | 177 | 3.4 |
| 1-02-0263L | 3M9793R | 42000 | 14.2 | 20.4 | 5.9 * 5.9 | 799 | 1350 | 695 | 979 | 1190 | 189 | 3.7 |
| 1-02-1012L | 3M9793R | 10000 | 14 | 21.97 | 5.6 * 5.9 | 528 | 1100 | 553 | 784 | 976 | 172 | 3.2 |
| 1-02-1012L | 3M9793R | 23000 | 14.9 | 21.65 | 5.7 * 5.8 | 600 | 1190 | 603 | 693 | 951 | 184 | 3.3 |
| 1-02-1012L | 3M9793R | | 13.5 | 21.66 | 5.9 * 6.0 | 766 | 1500 | 689 | 865 | 1300 | 209 | 3.3 |

No changes were observed in the focal distance, focal length and/or the transducer diameter.

What we claim is:

1. A vibration delivery system comprising:
at least one vibrating element;
an acoustic coupling interface; and
a protective coating on said acoustic coupling interface.

2. The system according to claim 1, wherein said protective coating is a chemically protective coating.

3. The system according to claim 1, wherein said protective coating is composed of PVC coating.

4. The system according to claim 1, wherein said protective coating is composed of polyolefin film.

5. The system according to claim 1, wherein said protective coating is applied by adhering to the acoustic coupling interface.

6. The system according to claim 1, wherein said protective coating is mechanically applied to the acoustic coupling interface.

7. The system according to claim 1, wherein said coating is acoustically matched to said acoustic coupling interface.

8. The system according to claim 1, wherein said acoustic coupling interface comprises a polyurethane membrane.

9. A coating for an acoustic coupling interface comprising:
a protective material adapted to inhibit a reaction between said acoustic coupling interface and an external surface.

10. The coating of claim 9, wherein the protective material is a chemically protective material.

11. The coating of claim 9, wherein the reaction is a chemical reaction.

12. The coating of claim 9, wherein said external surface comprises a substrate, a tissue, a substance, an interposer or any combination thereof.

13. The coating of claim 12 wherein said tissue comprises skin tissue, adipose tissue or any combination thereof.

14. The coating of claim 12, wherein said interposer comprises a lubricant.

15. The coating of claim 14, wherein said lubricant comprises castor oil.

16. The coating of claim 9, wherein said protective material comprises PVC coating.

17. The coating of claim 9, wherein said protective material comprises a polyolefin film.

18. The coating of claim 9, wherein said protective material is applied by adhering to said acoustic coupling interface.

19. The coating of claim 9, wherein said protective material is mechanically applied to said acoustic coupling interface.

20. The coating of claim 9, wherein said acoustic coupling interface comprises a polyurethane membrane.

21. A system for selectively damaging fat cells, the system comprising:
at least one vibrating energy transducer;
an acoustic coupling interface; and
a protective coating on said acoustic coupling interface.

22. The system according to claim 21, wherein said protective coating is a chemically protective coating.

23. A method for ameliorating chemical interaction between an acoustic coupling interface and an interposer, the method comprising:
applying a chemically protective coating on said acoustic coupling interface.

24. The method according to claim 23, wherein said interposer comprises a lubricant.
